(19) United States
(12) Patent Application Publication
(10) Pub. No.: US 2026/0253691 A1
(43) Pub. Date: Aug. 27, 2026

(54) SYSTEM

(71) Applicant: SoftBank Group Corp., Tokyo (JP)

(72) Inventor: Mitsue ECHIZEN, Tokyo (JP)

(73) Assignee: SoftBank Group Corp., Tokyo (JP)

(21) Appl. No.: 19/537,673

(22) Filed: Feb. 12, 2026

(30) Foreign Application Priority Data
Feb. 21, 2025 (JP) 2025-027094

(51) Int. Cl. G16H 10/60; G06F 40/20; G06F 40/40; G06N 5/04; G16H 15/00; G16H 50/20

(52) U.S. Cl. CPC G16H 10/60; G06F 40/20; G06F 40/40; G16H 15/00; G16H 50/20; G06N 5/04

(57) ABSTRACT

The system according to the embodiment comprises a collection unit, an analysis unit, a suggestion unit, and a display unit. The collection unit collects lifelog data and GPS data. The analysis unit analyzes the data collected by the collection unit and evaluates the user's physical condition. The suggestion unit proposes a recipe based on the physical condition evaluated by the analysis unit. The display unit displays a price list of ingredients required for the recipe proposed by the suggestion unit.

### CROSS-REFERENCE TO RELATED APPLICATION

The present application claims priority to and incorporates by reference the entire contents of Japanese Patent Application No. 2025-027094 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, recipe suggestions based on the user's physical condition and the provision of price information for ingredients have not been sufficiently performed, leaving room for improvement.

### SUMMARY OF THE INVENTION

The system according to the embodiment comprises a collection unit, an analysis unit, a suggestion unit, and a display unit. The collection unit collects lifelog data and GPS data. The analysis unit analyzes the data collected by the collection unit and evaluates the user's physical condition. The suggestion unit proposes a recipe based on the physical condition evaluated by the analysis unit. The display unit displays a price list of ingredients required for the recipe proposed by the suggestion unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

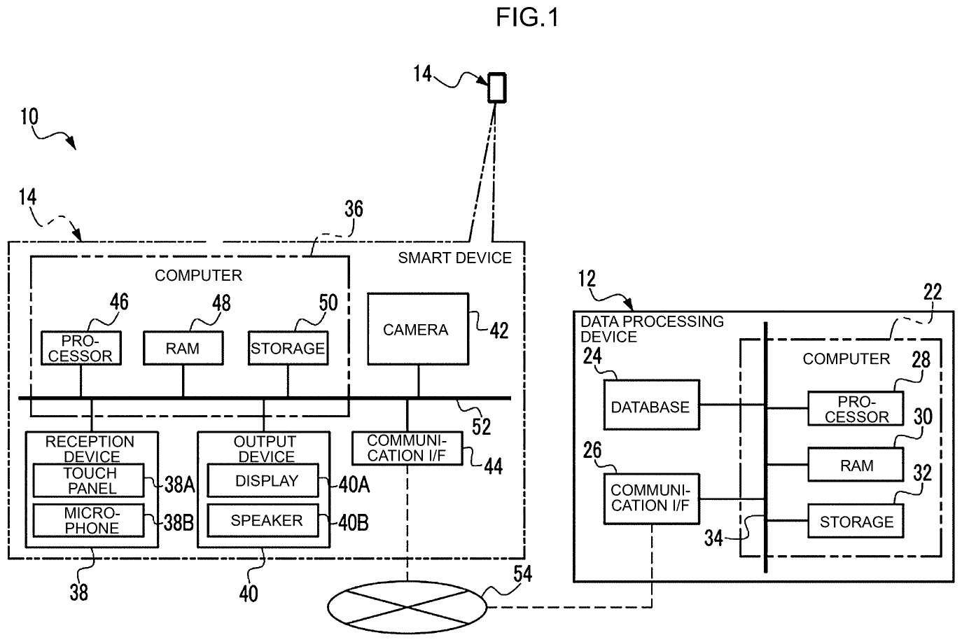

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

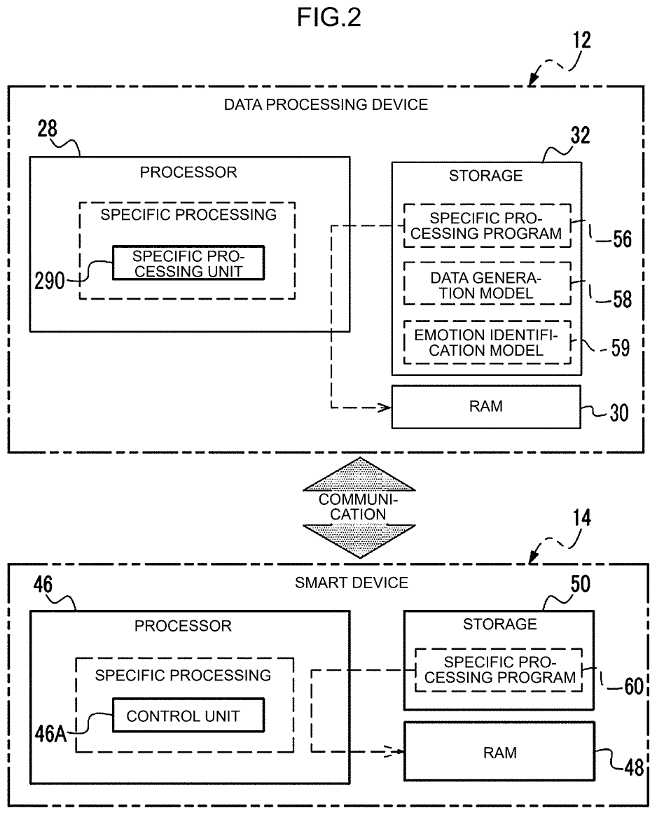

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The health management system according to the embodiment of the present invention is a system that links lifelog data and GPS, proposes recommended recipes based on the most recent physical condition data, and displays a price list of ingredients within the user's living area. This health management system collects the user's lifelog data (for example, sleep duration, amount of exercise, meal contents, heart rate, etc.) and GPS data, analyzes them using AI, and evaluates the user's recent physical condition. For example, it detects conditions such as sleep deprivation or lack of exercise. Based on this evaluation, the AI proposes the optimal recipe to the user, such as recipes using ingredients effective for fatigue recovery. Furthermore, the system collects and displays a price list of ingredients required for the proposed recipe from stores within the user's living area, enabling the user to purchase ingredients at the optimal price. When going out, the AI analyzes the menus of restaurants near the current location and proposes restaurants that offer menus suitable for the user's physical condition, such as restaurants offering easily digestible menus. In addition, the system provides a mechanism for awarding points when the user consumes a healthy meal, thereby motivating the user to maintain a healthy diet. Thus, the health management system can propose optimal recipes and display a price list of ingredients based on the user's physical condition. Specifically, the health management system is composed of multiple hardware and software modules such as a collection unit, analysis unit, suggestion unit, and display unit. The collection unit collects multidimensional time-series data such as sleep duration (e.g., 7 hours, 5 hours), amount of exercise (e.g., 10,000 steps per day, 30 minutes of running), meal contents (e.g., eggs and toast for breakfast, salad and chicken for lunch), and heart rate (e.g., 70 bpm at rest, 120 bpm during exercise) from wearable devices or smartphone sensors, with a resolution of one day or one minute. GPS data is acquired as vector data including latitude, longitude, altitude, and timestamp. The analysis unit receives these data as input tensors (e.g., 3D tensors of user×time×number of features) and uses convolutional neural networks (CNN), recurrent neural networks (RNN), or Transformer-based time-series analysis models to output the user's physical condition (e.g., fatigue score 0.8, stress level 0.6, sleep deprivation label 1). Examples of AI model inputs include “sleep duration array for the past 7 days,” “daily exercise vector,” “categorical encoding of meal contents,” and “time-series array of heart rate.” Examples of AI model outputs include “fatigue score (0.0-1.0),” “estimated stress level (low, medium, high),” and “recommended nutrients (protein, vitamin C).” The suggestion unit receives the output of the analysis unit, searches for recipes that meet the conditions from a recipe database, and applies recommendation algorithms (e.g., collaborative filtering, content-based filtering, reinforcement learning-based recommendation) to select the optimal recipe. For example, if the fatigue level is high, recipes rich in vitamin B are prioritized, and if sleep deprivation is detected, easily digestible menus are prioritized. Examples of recipe suggestions include “chicken breast and broccoli salad” and “salmon foil bake.” The display unit generates a list of required ingredients for the proposed recipe, obtains price information (e.g., a pack of eggs 200 yen, 100 g chicken breast 120 yen) in real time from store information databases or online APIs within the living area, and displays it on the user's device in list or graph format. Furthermore, when going out, the display unit collects menu information from restaurant databases near the current location based on GPS data, analyzes the menu contents using natural language processing AI (e.g., large language models), and recommends restaurants suitable for the user's physical condition (e.g., Japanese restaurants offering easily digestible food, restaurants offering low-calorie menus). Examples of AI inputs include “text data of restaurant menus,” “user physical condition score,” and “current location information,” and examples of outputs include “recommended restaurant list” and “health suitability score for each store.” In addition, when the user selects a healthy meal, the point management module matches the meal record data and physical condition evaluation results, and automatically awards points according to the achievement of nutritional balance and calorie restriction. Points are accumulated in the user's account and can be used for exchanging benefits or receiving health advice. This series of processing is realized by advanced application of computer technology, such as automatic analysis of high-dimensional data, integration of multiple data sources, application of unconventional recommendation algorithms, and real-time linkage of price information, which differs from conventional manual recording, judgment, and recommendation. As a result, technical effects such as significant improvement in processing speed, increased accuracy of physical condition evaluation and recipe recommendation, optimization of user experience, and efficiency of communication and computation load are obtained. Specific application fields include personal health management applications, employee health support systems for companies, lifestyle guidance support for medical institutions, and health promotion services for local governments.

The health management system according to the embodiment comprises a collection unit, an analysis unit, a suggestion unit, and a display unit. The collection unit collects lifelog data and GPS data. Lifelog data includes, for example, sleep duration, amount of exercise, meal contents, heart rate, and the like, but is not limited to such examples. The collection unit can collect these data using, for example, wearable devices or smartphones. GPS data provides user location information and is used to obtain information about stores and restaurants within the living area. The analysis unit analyzes the data collected by the collection unit and evaluates the user's physical condition. The analysis unit can analyze the data using AI, for example, and evaluate the user's physical condition. For example, AI can evaluate the user's fatigue level and stress level based on sleep data and exercise data. The suggestion unit proposes recipes based on the physical condition evaluated by the analysis unit. The suggestion unit can use AI, for example, to propose the optimal recipe to the user. For example, AI can propose recipes using ingredients effective for fatigue recovery. The display unit displays a price list of ingredients required for the recipe proposed by the suggestion unit. The display unit can collect online price information and obtain and display price information from stores within the user's living area. This enables the user to purchase ingredients at the optimal price. Furthermore, the display unit can cooperate with store databases to collect and display price information, allowing the user to obtain the latest price information. Thus, the health management system according to the embodiment can propose optimal recipes and display a price list of ingredients based on the user's physical condition. Specifically, the collection unit collects multidimensional data such as sleep duration (e.g., 7.5 hours, 5.2 hours), amount of exercise (e.g., 10,000 steps per day, 350 kcal burned), meal contents (e.g., oatmeal for breakfast, chicken salad for lunch), and heart rate (e.g., 65 bpm at rest, 120 bpm during exercise) for each user via wearable devices (e.g., accelerometer, heart rate sensor, blood pressure monitor) or smartphone applications, recorded as time-series data. GPS data is acquired as vector data including latitude, longitude, altitude, and timestamp every minute, and is used to identify the user's movement history and living area. The analysis unit preprocesses the data received from the collection unit as a 3D tensor (user×time ×number of features), and uses convolutional neural networks (CNN), recurrent neural networks (RNN), or Transformer-based time-series analysis models to estimate the user's physical condition (e.g., fatigue score 0.7, stress level 0.5, sleep deprivation label 1). Examples of AI model inputs include “sleep duration array for the past 7 days,” “daily exercise vector,” “categorical encoding of meal contents,” and “time-series array of heart rate,” and examples of outputs include “fatigue score (0.0-1.0),” “estimated stress level (low, medium, high),” and “recommended nutrients (protein, vitamin C).” The suggestion unit receives the output of the analysis unit, searches for recipes that meet the conditions from a recipe database, and applies recommendation algorithms (e.g., collaborative filtering, content-based filtering, reinforcement learning-based recommendation) to select the optimal recipe. For example, if the fatigue level is high, recipes rich in vitamin B are prioritized, and if sleep deprivation is detected, easily digestible menus are prioritized. Examples of recipe suggestions include “chicken breast and broccoli salad” and “salmon foil bake.” The display unit generates a list of required ingredients for the proposed recipe, obtains price information (e.g., a pack of eggs 200 yen, 100 g chicken breast 120 yen) in real time from store information databases or online APIs within the living area, and displays it on the user's device in list or graph format. Furthermore, the display unit can cooperate with store databases and APIs to obtain and display the latest price information. This series of processing is realized by advanced application of computer technology, such as automatic analysis of high-dimensional data, integration of multiple data sources, application of unconventional recommendation algorithms, and real-time linkage of price information, which differs from conventional manual recording, judgment, and recommendation. As a result, technical effects such as significant improvement in processing speed, increased accuracy of physical condition evaluation and recipe recommendation, optimization of user experience, and efficiency of communication and computation load are obtained. Specific application fields include personal health management applications, employee health support systems for companies, lifestyle guidance support for medical institutions, and health promotion services for local governments.

The collection unit can collect lifelog data such as sleep duration, amount of exercise, meal contents, heart rate, and blood pressure. For example, the collection unit can use a wearable device to measure sleep duration. For example, the collection unit records the time from when the user goes to bed until waking up and collects it as sleep data. The collection unit can use an accelerometer to measure the amount of exercise. For example, the collection unit records the user's number of steps and exercise time and collects them as exercise data. Furthermore, the collection unit can use an application for the user to record meal contents. For example, the collection unit records the ingredients eaten and calorie intake and collects them as meal data. The collection unit can use a heart rate sensor to measure heart rate. For example, the collection unit measures the user's heart rate in real time and collects it as heart rate data. The collection unit can use a blood pressure monitor to measure blood pressure. For example, the collection unit periodically measures the user's blood pressure and collects it as blood pressure data. Thus, the collection unit can collect the user's lifelog data in detail. Specifically, the collection unit collects multidimensional time-series data for each user at a resolution of one minute or one day via wearable devices (e.g., accelerometer, heart rate sensor, blood pressure monitor, skin temperature sensor, SpO2 sensor) or smartphone applications. The collection unit records sleep duration data such as “bedtime (e.g., 23:00), wake-up time (e.g., 6:30), total sleep duration (e.g., 7.5 hours),” collects exercise data such as “number of steps (e.g., 10,000 steps per day), calories burned (e.g., 350 kcal), exercise intensity (e.g., METs value),” records meal contents data such as “meal time (e.g., breakfast at 7:30), ingredient list (e.g., eggs, toast, salad), calorie intake (e.g., 450 kcal), nutrient distribution (e.g., protein 20 g, fat 10 g, carbohydrate 60 g),” obtains heart rate data such as “resting heart rate (e.g., 65 bpm), heart rate during exercise (e.g., 120 bpm), heart rate variability (e.g., SDNN value)” in real time, and periodically records blood pressure data such as “systolic blood pressure (e.g., 120 mmHg), diastolic blood pressure (e.g., 80 mmHg), measurement time.” The collection unit stores these data in a structured database keyed by user ID, timestamp, and data type, and can apply preprocessing algorithms (e.g., moving average, outlier removal) for missing value imputation and anomaly detection. By automating data collection, the collection unit achieves real-time and highly accurate data acquisition, greatly improving data consistency and reliability compared to conventional manual input or paper records. As a technical effect, the collection unit ensures comprehensiveness and diversity of data by integrally managing data from multiple biosensors and applications, contributing to improved accuracy of subsequent AI analysis for physical condition estimation and recipe recommendation. In addition, automation and high-frequency data collection enable immediate detection of changes in the user's health status, allowing for early health risk detection and personalized health support. Specific application fields include personal health management applications, employee health monitoring for companies, remote health observation for medical institutions, and health promotion programs for local governments.

The display unit can collect and display online price information. For example, the display unit collects online price information via the Internet. For example, the display unit obtains price information from online shopping sites or price comparison sites and displays it to the user. The display unit can set the frequency of price information updates. For example, the display unit can be set to update price information daily. Furthermore, the display unit can provide an interface for visually displaying price information. For example, the display unit displays price information as graphs or charts, allowing the user to grasp price fluctuations at a glance. Thus, the display unit can collect and display online price information. Specifically, the display unit periodically obtains product price data (e.g., product ID, product name, price, stock status, store name, acquisition time) in JSON or CSV format from multiple online shopping sites or price comparison platforms via online APIs (e.g., RESTful API, GraphQL API). The display unit can flexibly change the price information acquisition interval (e.g., every hour, every day) and target product categories (e.g., ingredients, health foods, daily necessities) based on user settings or system policies. The display unit stores the acquired price data in an internal database and, by comparing with past price history, can apply trend analysis and lowest price detection algorithms (e.g., moving average method, outlier detection). As a user interface, the display unit provides multiple display modes such as list format (e.g., table of product name, price, store name), graph format (e.g., time-series line graph, heat map), and chart format (e.g., pie chart, bar graph), allowing users to intuitively grasp price trends and price differences between stores. The display unit is equipped with automatic price information updates and alert functions (e.g., notification when price falls below a certain value), supporting users to purchase products at the optimal timing. As a technical effect, the display unit realizes significant improvements in efficiency, accuracy, and user convenience in information acquisition compared to conventional manual searches or paper-based price comparisons, through automatic collection and visualization of online price information. In addition, real-time price fluctuation monitoring enables users to immediately grasp market trends, optimize cost reduction, and purchasing strategies. Specific application fields include personal shopping support apps, corporate purchasing management systems, distribution industry price survey tools, and consumer support services for local governments.

The display unit can cooperate with store databases to collect and display price information. For example, the display unit cooperates with store databases via APIs. For example, the display unit obtains price information from store databases and displays it to the user. The display unit can set the frequency of price information updates. For example, the display unit can be set to update price information in real time. Furthermore, the display unit can provide an interface for visually displaying price information. For example, the display unit displays price information in list or graph format, allowing the user to grasp price fluctuations at a glance. Thus, the display unit can cooperate with store databases to collect and display price information. Specifically, the display unit cooperates with product databases managed by each store (e.g., SQL database, NoSQL database) and APIs (e.g., REST API, WebSocket) to obtain product price information (e.g., product ID, product name, price, stock quantity, update time) in real time or periodically. The display unit stores the acquired price information in an internal cache database and, in conjunction with past price history and stock status, can generate trend analysis and out-of-stock alerts. As a user interface, the display unit provides multiple display modes such as list format (e.g., table of product name, price, store name), graph format (e.g., time-series line graph, heat map), and map-linked display (e.g., mapping store locations and price information), allowing users to intuitively grasp price differences and price trends for each store. The display unit is equipped with automatic price information updates and user customization functions (e.g., display only prices of specific stores, price fluctuation alerts), supporting users to make optimal purchasing decisions. As a technical effect, the display unit realizes significant improvements in efficiency, accuracy, and user convenience in information acquisition compared to conventional manual surveys or paper-based price comparisons, through real-time price information acquisition and visualization via API cooperation with store databases. In addition, integrated management and analysis of price information from multiple stores enables users to immediately select the optimal purchasing destination, optimize cost reduction, and purchasing strategies. Specific application fields include personal shopping support apps, corporate purchasing management systems, distribution industry price survey tools, and consumer support services for local governments.

The suggestion unit can collect menu information of restaurants within a radius of 500 meters from the current location when going out and propose restaurants that provide menus suitable for the user's physical condition. For example, the suggestion unit identifies the user's current location using GPS data. For example, the suggestion unit collects information on restaurants within a radius of 500 meters from the user's current location. The suggestion unit can obtain menu information of restaurants via the Internet from restaurant websites or menu information. For example, the suggestion unit analyzes restaurant menu information and identifies restaurants that provide menus suitable for the user's physical condition. Furthermore, the suggestion unit can use algorithms to propose restaurants based on the user's physical condition. For example, the suggestion unit can use an AI model that takes the user's physical condition data as input and outputs the optimal restaurant to propose restaurants. Thus, the suggestion unit can propose restaurants that provide menus suitable for the user's physical condition when going out. Specifically, the suggestion unit identifies the user's current location (latitude, longitude, altitude, timestamp) with high accuracy using GPS sensors or smartphone location APIs, and obtains a list of restaurants within a radius of 500 meters (e.g., store ID, store name, address, genre, business hours) from geographic information databases or restaurant information APIs (e.g., restaurant search service API). The suggestion unit collects menu information for each restaurant (e.g., menu name, ingredient list, calories, nutrients, price, allergen information) via web scraping or API, and analyzes menu contents using natural language processing AI (e.g., large language models, BERT-based models). The suggestion unit inputs the user's physical condition data (e.g., fatigue score 0.8, stress level 0.6, recommended nutrient list) as an input tensor to the AI model and outputs a health suitability score (e.g., 0.0-1.0) for each restaurant menu. Examples of AI model inputs include “user physical condition score vector,” “menu nutrient vector,” and “current location information vector,” and examples of outputs include “recommended restaurant list” and “health suitability score for each store.” The suggestion unit applies recommendation algorithms (e.g., content-based filtering, reinforcement learning-based recommendation) and prioritizes restaurants most suitable for the user's physical condition. As a subsequent process, the suggestion unit sends the recommended restaurant list to the display unit, which can present it on the user's device in map-linked or ranking format. As a technical effect, the suggestion unit automatically analyzes the user's physical condition data and restaurant menu information in a high-dimensional vector space, realizing personalized and scientifically grounded restaurant recommendations, which differs from conventional manual searches or experience-based recommendations. This enables users to select optimal meals for their health even when going out, contributing to health maintenance and disease prevention. Specific application fields include personal health support apps, restaurant navigation for tourists, corporate welfare support, and health promotion services for local governments.

The suggestion unit can provide a mechanism for awarding points when a healthy meal is consumed. For example, the suggestion unit provides an application for recording when the user consumes a healthy meal. For example, the suggestion unit records the user's meal contents and awards points when a healthy meal is consumed. The suggestion unit can set criteria for awarding points. For example, the suggestion unit can set criteria for awarding points based on nutritional balance or calorie restriction. Furthermore, the suggestion unit can provide a system for managing points. For example, the suggestion unit provides a system for managing points earned by the user and for receiving benefits using points. Thus, the suggestion unit can provide a mechanism for awarding points when a healthy meal is consumed. Specifically, the suggestion unit stores meal data (e.g., ingredient list, calorie intake, nutrient distribution, meal time) obtained from applications for recording meal contents or wearable devices in an internal database, and the AI analysis unit calculates a health evaluation of the meal contents (e.g., nutritional balance score, calorie suitability, recommended nutrient fulfillment rate). The suggestion unit can set multiple health indicators as criteria for awarding points, such as nutritional balance (e.g., protein, fat, carbohydrate ratio), calorie restriction (e.g., less than 500 kcal per meal), and intake of specific nutrients (e.g., at least 100 mg vitamin C). The suggestion unit applies a point awarding algorithm (e.g., score-linked, achievement-level) based on the AI model output (e.g., health score 0.85, nutritional balance achievement label 1) and automatically adds points to the user's account. The suggestion unit is equipped with a point management module for unified management of each user's point history, benefit exchange history, point expiration date, etc., enabling users to exchange points for health advice or benefits (e.g., discount coupons, health food samples). As a technical effect, the suggestion unit systematizes automatic evaluation of healthy behavior and incentive awarding, greatly improving objectivity, transparency, and immediacy of evaluation compared to conventional self-reporting or manual aggregation. In addition, flexible changes to point awarding criteria and benefit content continuously promote changes in user health behavior and maximize health promotion effects. Specific application fields include personal health management apps, corporate health management support, health incentive programs for insurance companies, and health point projects for local governments.

The collection unit can estimate the user's emotion and adjust the timing of lifelog data collection based on the estimated emotion of the user. For example, the collection unit estimates the user's emotion using emotion recognition algorithms. For example, the collection unit analyzes the user's facial expressions and voice data to estimate emotion. Furthermore, the collection unit can adjust the timing of lifelog data collection based on the estimated emotion. For example, if the user is feeling stressed, the collection unit collects lifelog data during relaxed periods. If the user is relaxed, the collection unit can collect lifelog data during active periods. If the user is tired, the collection unit can prioritize collecting data during rest. Thus, the collection unit can adjust the timing of lifelog data collection based on the user's emotion. Emotion estimation is realized using, for example, emotion engines or generative AI with emotion estimation functions. Generative AI includes text generation AI (e.g., LLM) and multimodal generative AI, but is not limited thereto. Specifically, the collection unit simultaneously acquires the user's facial image data (e.g., 128×128 pixel RGB tensor), voice waveform data (e.g., 1 minute of 16 kHz sampled audio), and text data (e.g., user diary or SNS posts), inputs them as multimodal input tensors to an emotion estimation AI model. The emotion estimation AI model adopts a multimodal architecture combining facial expression feature extraction by CNN, voice emotion recognition by RNN or Transformer-based models, and text emotion analysis by natural language processing models (e.g., BERT-based). Examples of AI model inputs include “smiling face image,” “angry face image,” “calm voice waveform,” “high-pitched fast speech waveform,” “positive diary text,” and “negative SNS post.” Examples of AI model outputs include “emotion label (e.g., stress, relaxation, fatigue, joy, sadness)” and “emotion score (e.g., stress 0.7, relaxation 0.2, fatigue 0.1).” The collection unit controls the timing of lifelog data collection (e.g., heart rate, sleep duration, amount of exercise, meal contents, blood pressure) based on the output of the emotion estimation AI model. For example, if the stress score is high, data collection is concentrated during nighttime or holidays when the user is estimated to be relaxed. If the relaxation score is high, data collection is performed during active periods (e.g., morning or after exercise). If the fatigue score is high, biosignals during rest or sleep (e.g., sleep depth, heart rate variability) are preferentially collected. These controls are realized by the scheduler module of the collection unit automatically adjusting the collection timing based on AI output values. As a technical effect, the collection unit dynamically optimizes collection timing according to the user's emotional state, achieving high-precision and efficient data acquisition responsive to changes in user state, compared to conventional fixed-time or uniform collection. This improves the accuracy of physical condition estimation and health risk detection, and reduces unnecessary data collection and computation/communication load. Specific application fields include personal health management applications, stress management support systems, employee mental health monitoring for companies, and remote health observation for medical institutions.

The collection unit can analyze the user's past lifelog data and select a collection method. For example, the collection unit analyzes the user's past lifelog data using AI. For example, the collection unit analyzes the user's past sleep data and determines the optimal collection timing. The collection unit can also analyze the user's exercise data and prioritize data collection after exercise. Furthermore, the collection unit can analyze the user's meal data and collect data after meals. Thus, the collection unit can select the optimal collection method by analyzing the user's past lifelog data. Specifically, the collection unit stores multidimensional arrays such as sleep duration data (e.g., daily bedtime, wake-up time, total sleep duration), exercise data (e.g., number of steps, calories burned, exercise intensity), and meal contents data (e.g., meal time, calorie intake, nutrient distribution) for each user in a structured database keyed by user ID and timestamp. The collection unit inputs these past data as input tensors (e.g., 3D tensor of user×date×number of features) to an AI analysis model. The AI model uses recurrent neural networks (RNN) or Transformer-based time-series pattern recognition models to extract lifestyle rhythms and behavioral patterns for each user. Examples of AI model inputs include “sleep duration array for the past 30 days,” “weekly exercise vector,” and “time-series array of meal times.” Examples of AI model outputs include “optimal data collection timing (e.g., every day at 22:00, within 30 minutes after exercise, within 1 hour after meals)” and “collection frequency (e.g., 3 times a day, additional collection on exercise days).” Based on the output of the AI model, the collection scheduler module automatically adjusts data collection timing and frequency. For example, if the user has a nocturnal lifestyle, sleep data is intensively collected at night, and if the user has an exercise habit, biosignals (e.g., heart rate, blood pressure) are preferentially collected immediately after exercise. If the meal pattern is irregular, data collection is performed in accordance with the input timing of the meal recording app. As a technical effect, the collection unit automatically generates personalized collection strategies based on each user's past data, greatly improving the comprehensiveness, usefulness, and reliability of data compared to conventional uniform collection or manual settings. In addition, unnecessary data collection is suppressed, and optimization of computation and communication resources is achieved. Specific application fields include personal health management apps, lifestyle disease prevention programs, corporate health management support, and remote monitoring for medical institutions.

The collection unit can perform filtering based on the user's current living situation and areas of interest at the time of lifelog data collection. For example, the collection unit analyzes the user's current living situation and areas of interest using AI. For example, if the user is on a diet, the collection unit prioritizes collecting data related to meal contents. If the user has just started exercising, the collection unit can focus on collecting data related to amount of exercise. Furthermore, if the user is about to undergo a health checkup, the collection unit can collect data related to heart rate and blood pressure. Thus, by performing filtering based on the user's current living situation and areas of interest, the collection unit can collect more relevant data. Specifically, the collection unit integrates the user's profile information (e.g., age, gender, medical history), recent lifelog data (e.g., weight change, exercise start date, scheduled health checkup date), and areas of interest set by the user in the application (e.g., diet, muscle strengthening, blood pressure management) as feature vectors and inputs them to an AI model. The AI model uses content-based filtering algorithms or reinforcement learning-based filtering models to automatically select the data types most relevant to the user's current goals and interests. Examples of AI model inputs include “diet goal set,” “3 days since starting exercise,” and “2 weeks until health checkup.” Examples of AI model outputs include “priority data types for collection (e.g., meal contents, calorie intake, amount of exercise, heart rate, blood pressure)” and “collection priority score (e.g., meal contents 0.9, amount of exercise 0.8, blood pressure 0.7).” Based on the output of the AI model, the data collection module of the collection unit intensively collects high-priority data types and reduces the collection frequency of low-priority data. For example, for users on a diet, meal contents and calorie intake are collected at high frequency, and for users who have just started exercising, amount of exercise and heart rate are intensively collected. For users before a health checkup, continuous monitoring of blood pressure and heart rate is performed. As a technical effect, the collection unit realizes dynamic data filtering according to the user's living situation and areas of interest, efficiently acquiring highly useful data for the user compared to conventional uniform collection, and contributing to improved accuracy of data analysis and health support. In addition, unnecessary data collection is suppressed, and optimization of storage and communication resources is achieved. Specific application fields include personalized health management apps, diet support services, fitness monitoring, and lifestyle guidance support for medical institutions.

The collection unit can estimate the user's emotion and determine the priority of lifelog data to be collected based on the estimated emotion of the user. For example, the collection unit estimates the user's emotion using emotion recognition algorithms. For example, the collection unit analyzes the user's facial expressions and voice data to estimate emotion. Furthermore, the collection unit can determine the priority of lifelog data to be collected based on the estimated emotion. For example, if the user is feeling stressed, the collection unit prioritizes collecting heart rate and sleep data. If the user is relaxed, the collection unit can prioritize collecting exercise data. If the user is tired, the collection unit can prioritize collecting data related to meal contents. Thus, by determining the priority of lifelog data to be collected based on the user's emotion, the collection unit can prioritize collecting more important data. Emotion estimation is realized using, for example, emotion engines or generative AI with emotion estimation functions. Generative AI includes text generation AI (e.g., LLM) and multimodal generative AI, but is not limited thereto. Specifically, the collection unit simultaneously acquires the user's facial images (e.g., 128×128 pixel RGB images), voice data (e.g., 1 minute of audio waveform), and text data (e.g., SNS posts or diary text), and inputs them to a multimodal AI model. The emotion estimation AI model adopts an architecture combining image feature extraction by CNN and emotion analysis of voice and text by RNN or Transformer. Examples of AI model inputs include “smiling face image,” “angry face image,” “calm voice,” “high-pitched voice,” “positive text,” and “negative text.” Examples of AI model outputs include “emotion label (stress, relaxation, fatigue)” and “emotion score (stress 0.8, relaxation 0.1, fatigue 0.1).” Based on the output of the AI model, the collection unit automatically determines the priority of lifelog data to be collected (e.g., heart rate, sleep duration, amount of exercise, meal contents, blood pressure). For example, if the stress score is high, heart rate and sleep data are collected with the highest priority; if the relaxation score is high, exercise data is prioritized; if the fatigue score is high, meal contents data is intensively collected. These priorities are dynamically adjusted by the data collection scheduler of the collection unit based on AI output values. As a technical effect, the collection unit optimizes the priority of data collection according to the user's emotional state, enabling acquisition of important data responsive to changes in the user's health status, improving the accuracy of physical condition estimation and health risk detection, and achieving efficiency in computation and communication load compared to conventional uniform collection. Specific application fields include personal health management apps, stress management support, employee health monitoring for companies, and remote health observation for medical institutions.

The collection unit can preferentially collect highly relevant data within a radius of 1 kilometer from the user's current location by considering the user's geographic location information at the time of lifelog data collection. For example, the collection unit identifies the user's geographic location using GPS data. For example, the collection unit preferentially collects highly relevant data within a radius of 1 kilometer from the user's current location. If the user is traveling, the collection unit can prioritize collecting data related to travel distance and number of steps. If the user is at home, the collection unit can prioritize collecting data related to sleep and meal contents. Thus, by considering the user's geographic location information, the collection unit can preferentially collect more appropriate data. Specifically, the collection unit acquires the user's current location (latitude, longitude, altitude, timestamp) with high accuracy using GPS sensors or smartphone location APIs, and identifies the user's living area and movement history in cooperation with geographic information databases. The collection unit obtains facility information such as stores, restaurants, parks, and gyms within a radius of 1 kilometer from the current location, and also collects facility usage history and surrounding environmental data (e.g., temperature, humidity, noise level). The AI model receives the user's current location, movement pattern, and facility usage history as input vectors and outputs the priority of relevant lifelog data types (e.g., travel distance, number of steps, meal contents, sleep duration, heart rate). Examples of AI model inputs include “current location: tourist spot,” “travel distance: 5 km,” “facility usage: gym,” and “at home.” Examples of AI model outputs include “priority data types for collection (e.g., travel distance and number of steps while traveling, sleep and meal contents at home)” and “collection priority score.” Based on the output of the AI model, the collection unit collects travel distance and number of steps at high frequency while traveling, and focuses on collecting sleep data and meal contents while at home. This enables optimal data collection according to the user's geographic location and behavioral situation. As a technical effect, the collection unit achieves high-precision data acquisition responsive to changes in the user's behavior and environment by dynamic data collection control utilizing geographic location information, improving the accuracy of physical condition estimation and health support, and achieving efficiency in computation and communication load compared to conventional uniform collection. Specific application fields include personal health management apps, health monitoring for travelers, employee health support for companies, and health promotion services for local governments.

The collection unit can analyze the user's social media activity and collect relevant data at the time of lifelog data collection. For example, the collection unit analyzes the user's social media activity using AI. For example, if the user posts about exercise on social media, the collection unit prioritizes collecting exercise data. If the user posts about meals, the collection unit can prioritize collecting data related to meal contents. If the user posts about stress, the collection unit can prioritize collecting heart rate and sleep data. Thus, by analyzing the user's social media activity, the collection unit can collect relevant data. Specifically, the collection unit automatically acquires public post data (e.g., text, images, videos, posting time, location tags) linked to the user ID from multiple social media platforms (e.g., microblogs, photo sharing services, video posting sites) via API or web scraping technology. The collection unit inputs the acquired post data to natural language processing AI (e.g., large language models, BERT-based models) or image recognition AI (e.g., CNN-based image classification models) to perform category classification of post contents (e.g., exercise, meals, stress, hobbies, travel) and emotion analysis (e.g., positive, negative, neutral). Examples of AI model inputs include “exercise-related post text (e.g., ran 5 km today),” “meal image (e.g., photo of salad),” and “stress-related post (e.g., work has been busy and tiring lately).” Examples of AI model outputs include “post category label (exercise, meals, stress)” and “emotion score (stress 0.7, positive 0.2, negative 0.1).” Based on the output of the AI model, the collection unit dynamically adjusts the priority of lifelog data collection. For example, if there are many exercise-related posts, the collection unit collects exercise data (e.g., number of steps, travel distance, calories burned) from accelerometers or GPS at high frequency; if there are many meal-related posts, the collection unit intensively collects ingredient, calorie, and nutrient data from meal recording apps; if stress-related posts are detected, the collection unit prioritizes collecting heart rate and sleep data (e.g., heart rate variability, sleep depth). These controls are realized by the scheduler module of the collection unit automatically optimizing collection timing and data types based on AI output values. As a technical effect, the collection unit realizes real-time analysis of external information sources such as the user's social media activity, enabling data collection responsive to changes in user interests and behavior, greatly improving the relevance, usefulness, and comprehensiveness of data compared to conventional uniform collection or manual settings. In addition, unnecessary data collection is suppressed, and optimization of computation and communication resources is achieved. Specific application fields include personalized health management apps, behavior change support services, employee health monitoring for companies, remote health observation for medical institutions, and health promotion programs for local governments.

The analysis unit can estimate the user's emotion and adjust the criteria for physical condition evaluation based on the estimated emotion of the user. For example, the analysis unit estimates the user's emotion using emotion recognition algorithms. For example, the analysis unit analyzes the user's facial expressions and voice data to estimate emotion. Furthermore, the analysis unit can adjust the criteria for physical condition evaluation based on the estimated emotion. For example, if the user is feeling stressed, the analysis unit performs physical condition evaluation with emphasis on stress level. If the user is relaxed, the analysis unit can perform physical condition evaluation with emphasis on overall health status. If the user is tired, the analysis unit can perform physical condition evaluation with emphasis on fatigue level. Thus, by adjusting the criteria for physical condition evaluation based on the user's emotion, the analysis unit can perform more accurate physical condition evaluation. Emotion estimation is realized using, for example, emotion engines or generative AI with emotion estimation functions. Generative AI includes text generation AI (e.g., LLM) and multimodal generative AI, but is not limited thereto. Specifically, the analysis unit simultaneously acquires the user's facial images (e.g., 128×128 pixel RGB images), voice data (e.g., 1 minute of 16 kHz sampled audio), and text data (e.g., diary text, SNS posts), and inputs them to a multimodal AI model. The emotion estimation AI model adopts an architecture combining image feature extraction by CNN and emotion analysis of voice and text by RNN or Transformer. Examples of AI model inputs include “smiling face image,” “angry face image,” “calm voice,” “high-pitched voice,” “positive text,” and “negative text.” Examples of AI model outputs include “emotion label (stress, relaxation, fatigue)” and “emotion score (stress 0.8, relaxation 0.1, fatigue 0.1).” Based on the output of the emotion estimation AI model, the analysis unit dynamically adjusts the weight parameters and evaluation criteria of the physical condition evaluation algorithm. For example, if the stress score is high, the physical condition evaluation model assigns weights to stress-related indicators (e.g., heart rate variability, sleep quality, cortisol level); if the relaxation score is high, overall health indicators (e.g., nutritional balance, amount of exercise, sleep duration) are emphasized; if the fatigue score is high, fatigue evaluation indicators (e.g., subjective fatigue score, muscle pain, rest time) are prioritized. These adjustments are realized by the evaluation logic of the analysis unit automatically changing weighting and threshold settings based on AI output values. As a technical effect, the analysis unit dynamically optimizes physical condition evaluation criteria according to the user's emotional state, realizing high-precision and personalized physical condition evaluation responsive to changes in user state, compared to conventional uniform evaluation or manual settings. This enables early detection of health risks and appropriate health support, contributing to improved user experience and efficiency in medical and health management. Specific application fields include personal health management apps, stress management support, employee health monitoring for companies, and remote health observation for medical institutions.

The analysis unit can improve the accuracy of physical condition evaluation by considering interrelationships among lifelog data (for example, the relationship between sleep duration and amount of exercise) during analysis. For example, the analysis unit analyzes interrelationships among lifelog data using AI. For example, the analysis unit analyzes the correlation between sleep data and heart rate data and reflects it in physical condition evaluation. The analysis unit can also analyze the correlation between exercise data and meal data and reflect it in physical condition evaluation. Furthermore, the analysis unit can analyze the correlation between stress level and sleep data and reflect it in physical condition evaluation. Thus, by considering interrelationships among lifelog data, the analysis unit can improve the accuracy of physical condition evaluation. Specifically, the analysis unit structures multidimensional lifelog data (e.g., sleep duration, amount of exercise, meal contents, heart rate, stress score) recorded as time-series data for each user as a 3D tensor (user×time×number of features) and inputs it to an AI analysis model. The AI analysis model adopts a multitask learning neural network (e.g., Transformer with multi-head attention, multi-stream RNN) to automatically extract interrelationships among features (e.g., sleep and exercise, meal and heart rate, stress and sleep) in high-dimensional space. Examples of AI model inputs include “sleep duration array for the past 30 days,” “weekly exercise vector,” “categorical encoding of meal contents,” “time-series array of heart rate,” and “time-series of stress score.” Examples of AI model outputs include “physical condition evaluation score (0.0-1.0),” “correlation coefficient matrix (e.g., sleep and exercise correlation 0.6, exercise and meal correlation 0.4),” and “health risk prediction label.” Based on the output of the AI model, the physical condition evaluation algorithm weights the interrelationships among features to calculate a comprehensive physical condition score. For example, if the correlation between sleep and exercise is high, both are evaluated simultaneously; if there is a strong negative correlation between stress and sleep, sleep quality deterioration during increased stress is considered a risk factor. These processes differ from conventional single-indicator evaluation or experience-based judgment, and greatly improve the accuracy and reliability of physical condition evaluation through automatic analysis of high-dimensional data and unconventional feature integration. As a technical effect, the analysis unit enables early detection of health risks and improved accuracy of personalized health support by automatically learning and utilizing complex interrelationships among lifelog data with AI. Specific application fields include personal health management apps, lifestyle disease prevention programs, corporate health management support, and remote monitoring for medical institutions.

The analysis unit can refer to the user's past physical condition data during analysis to perform physical condition evaluation. For example, the analysis unit refers to the user's past physical condition data using AI. For example, the analysis unit refers to the user's past sleep data and reflects it in the current physical condition evaluation. The analysis unit can also refer to the user's past exercise data and reflect it in the current physical condition evaluation. Furthermore, the analysis unit can refer to the user's past meal data and reflect it in the current physical condition evaluation. Thus, by referring to the user's past physical condition data, the analysis unit can perform more accurate physical condition evaluation. Specifically, the analysis unit stores past physical condition data (e.g., sleep duration, amount of exercise, meal contents, heart rate, stress score) recorded as time-series data for each user in a structured database keyed by user ID and timestamp, and inputs it to an AI analysis model. The AI analysis model uses recurrent neural networks (RNN) or Transformer-based time-series pattern recognition models to extract lifestyle rhythms and physical condition fluctuation patterns for each user. Examples of AI model inputs include “sleep duration array for the past 30 days,” “weekly exercise vector,” “time-series array of meal contents,” “time-series array of heart rate,” and “time-series of stress score.” Examples of AI model outputs include “current physical condition evaluation score (0.0-1.0),” “anomaly detection label (e.g., normal, caution, abnormal),” and “recommended action (e.g., recommend rest, increase exercise, improve diet).” Based on the output of the AI model, the analysis unit performs comparison and trend analysis with past data and reflects it in the current physical condition evaluation. For example, if there is a trend of sleep deprivation over the past week, the current fatigue evaluation is set higher; if the amount of exercise is increasing, it is evaluated as a sign of physical condition improvement. These processes differ from conventional one-off data evaluation or subjective judgment, and greatly improve the accuracy and reliability of physical condition evaluation through long-term data accumulation and AI-based pattern extraction. As a technical effect, the analysis unit realizes personalized physical condition evaluation utilizing each user's past data, contributing to early detection of health risks and improved accuracy of continuous health support. Specific application fields include personal health management apps, lifestyle disease prevention programs, corporate health management support, and remote monitoring for medical institutions.

The analysis unit can estimate the user's emotion and adjust the order of displaying the results of physical condition evaluation based on the estimated emotion of the user. For example, the analysis unit estimates the user's emotion using emotion recognition algorithms. For example, the analysis unit analyzes the user's facial expressions and voice data to estimate emotion. Furthermore, the analysis unit can adjust the order of displaying the results of physical condition evaluation based on the estimated emotion. For example, if the user is feeling stressed, the analysis unit displays the evaluation result of stress level first. If the user is relaxed, the analysis unit can display the evaluation result of overall health status first. If the user is tired, the analysis unit can display the evaluation result of fatigue level first. Thus, by adjusting the order of displaying the results of physical condition evaluation based on the user's emotion, the analysis unit enables easy-to-understand display for the user. Emotion estimation is realized using, for example, emotion engines or generative AI with emotion estimation functions. Generative AI includes text generation AI (e.g., LLM) and multimodal generative AI, but is not limited thereto. Specifically, the analysis unit simultaneously acquires the user's facial images (e.g., 128×128 pixel RGB images), voice data (e.g., 1 minute of 16 kHz sampled audio), and text data (e.g., diary text, SNS posts), and inputs them to a multimodal AI model. The emotion estimation AI model adopts an architecture combining image feature extraction by CNN and emotion analysis of voice and text by RNN or Transformer. Examples of AI model inputs include “smiling face image,” “angry face image,” “calm voice,” “high-pitched voice,” “positive text,” and “negative text.” Examples of AI model outputs include “emotion label (stress, relaxation, fatigue)” and “emotion score (stress 0.8, relaxation 0.1, fatigue 0.1).” Based on the output of the emotion estimation AI model, the analysis unit dynamically adjusts the order of displaying physical condition evaluation results. For example, if the stress score is high, the stress level evaluation is displayed at the top; if the relaxation score is high, the overall health status evaluation is displayed with priority; if the fatigue score is high, the fatigue evaluation is displayed first. These controls are realized by the interface control module of the display unit automatically optimizing the display order based on AI output values. As a technical effect, the analysis unit optimizes information presentation according to the user's emotional state, enabling users to quickly grasp important information responsive to changes in their own state, improving the efficiency of health management and user experience. Specific application fields include personal health management apps, stress management support, employee health monitoring for companies, and remote health observation for medical institutions.

The analysis unit can perform physical condition evaluation by considering the user's geographic distribution (for example, differences between urban and suburban areas) during analysis. For example, the analysis unit analyzes the user's geographic distribution using AI. For example, if the user is in a high-altitude area, the analysis unit can perform physical condition evaluation considering oxygen concentration. If the user is in an urban area, the analysis unit can perform physical condition evaluation considering air pollution. Furthermore, if the user is by the sea, the analysis unit can perform physical condition evaluation considering humidity. Thus, by considering the user's geographic distribution, the analysis unit can perform more accurate physical condition evaluation. Specifically, the analysis unit acquires the user's current location (latitude, longitude, altitude, timestamp) with high accuracy using GPS sensors or smartphone location APIs, and obtains the user's living area and environmental information (e.g., altitude, temperature, humidity, air pollution index, oxygen concentration, noise level) in cooperation with geographic information databases. The analysis unit integrates these geographic and environmental features with the user's lifelog data and inputs them to an AI analysis model (e.g., multimodal neural network). Examples of AI model inputs include “current location: altitude 2000 m,” “air pollution index: PM2.5=80,” “humidity: 80%,” and “temperature: 30°C.” Examples of AI model outputs include “physical condition evaluation score (0.0-1.0)” and “environmental risk label (e.g., high-altitude hypoxia risk, urban air pollution risk, seaside high humidity risk).” Based on the output of the AI model, the physical condition evaluation algorithm weights geographic and environmental factors to calculate a comprehensive physical condition score. For example, when staying at high altitude, the risk of fatigue due to decreased oxygen concentration is considered; when staying in urban areas, respiratory risk due to air pollution is evaluated; when staying by the sea, the risk of heatstroke due to high humidity is considered. These processes differ from conventional uniform evaluation or experience-based judgment, and greatly improve the accuracy and reliability of physical condition evaluation through automatic analysis of geographic and environmental data and unconventional feature integration. As a technical effect, the analysis unit realizes personalized physical condition evaluation responsive to changes in the user's geographic distribution and environment, contributing to early detection of health risks and improved accuracy of appropriate health support. Specific application fields include personal health management apps, health monitoring for travelers, employee health support for companies, remote health observation for medical institutions, and health promotion services for local governments.

The analysis unit can refer to relevant literature of the user during analysis to improve the accuracy of physical condition evaluation. For example, the analysis unit refers to relevant literature of the user using AI. For example, the analysis unit refers to the latest research papers related to the user's physical condition and reflects them in physical condition evaluation. The analysis unit can also refer to past literature related to the user's health status and reflect it in physical condition evaluation. Furthermore, the analysis unit can refer to specialized books related to the user's physical condition and reflect them in physical condition evaluation. Thus, by referring to relevant literature of the user, the analysis unit can improve the accuracy of physical condition evaluation. Specifically, the analysis unit automatically acquires summary data (e.g., title, abstract, keywords, publication year, author, citation count) of the latest research papers, past literature, and specialized books related to the user's physical condition and health status from online academic paper databases or medical guideline APIs. The analysis unit analyzes the acquired literature data using natural language processing AI (e.g., large language models, BERT-based models) and extracts findings relevant to physical condition evaluation (e.g., relationship between sleep deprivation and cardiovascular risk, health effects of specific nutrients, evidence of exercise and stress relief). Examples of AI model inputs include “abstract of the latest paper on sleep deprivation,” “meta-analysis results on exercise and stress,” and “guidelines on health effects of specific nutrients.” Examples of AI model outputs include “relevant findings label (e.g., sleep deprivation increases cardiovascular risk, exercise has stress relief effect),” “evidence level score (e.g., A, B, C),” and “recommended action (e.g., recommend extending sleep duration, recommend increasing exercise frequency).” Based on the output of the AI model, the physical condition evaluation algorithm weights the latest scientific findings and evidence and reflects them in physical condition evaluation. For example, if sleep deprivation is strongly associated with increased cardiovascular risk, the evaluation weight of sleep data is increased; if intake of specific nutrients is effective for health maintenance, it is reflected in the evaluation of meal contents. These processes differ from conventional evaluation relying on experience or subjective judgment, and realize objective and highly accurate physical condition evaluation based on the latest scientific evidence. As a technical effect, the analysis unit greatly improves the accuracy and reliability of physical condition evaluation and contributes to early detection of health risks and provision of appropriate health support through automatic reference to relevant literature and AI-based extraction of findings. Specific application fields include personal health management apps, diagnostic support for medical institutions, corporate health management support, and health promotion services for local governments.

The suggestion unit is capable of estimating the user's emotion and adjusting the method of recipe suggestion expression based on the estimated emotion. The suggestion unit, for example, estimates the user's emotion using an emotion recognition algorithm. For instance, the suggestion unit can analyze the user's facial expressions and voice data to estimate emotion. Furthermore, the suggestion unit can adjust the method of recipe suggestion expression based on the estimated emotion. For example, if the user is feeling stressed, the suggestion unit proposes simple and easy-to-understand recipes. If the user is relaxed, the suggestion unit can propose recipes with detailed procedures. Additionally, if the user is tired, the suggestion unit can propose recipes that are easy to prepare. By adjusting the method of recipe suggestion expression based on the user's emotion, the suggestion unit enables more appropriate recipe suggestions. Emotion estimation is realized, for example, by using an emotion engine or generative AI with emotion estimation functionality. Generative AI may include text generation AI (e.g., LLM) or multimodal generative AI, but is not limited to these examples. Specifically, the suggestion unit simultaneously acquires the user's facial image (e.g., 128×128 pixel RGB image), voice data (e.g., 1-minute audio sampled at 16 kHz), and text data (e.g., diary entries, SNS posts), and inputs these into a multimodal AI model. The emotion estimation AI model adopts an architecture that combines image feature extraction by CNN and emotion analysis of voice and text by RNN or Transformer. Examples of inputs to the AI model include “smiling face image,” “angry face image,” “calm voice,” “high-pitched voice,” “positive text,” and “negative text.” Examples of outputs from the AI model include “emotion labels (stress, relaxation, fatigue, joy, sadness)” and “emotion scores (stress 0.7, relaxation 0.2, fatigue 0.1).” Based on the output of the emotion estimation AI model, the suggestion unit dynamically sets suggestion expression parameters (e.g., level of detail in explanation, conciseness of steps, softness of tone) for the recipe suggestion generation AI (e.g., large language model). For example, if the stress score is high, the recipe explanation is generated as short sentences and mainly bullet points, and the suggestion text is limited to the minimum cooking steps. If the relaxation score is high, a long recipe including detailed explanations such as cooking tips, arrangement examples, and health effects of ingredients is generated. If the fatigue score is high, recipes with short cooking times and few steps are prioritized, and catchphrases such as “can be made in 5 minutes” or “only 3 ingredients” are automatically added. Examples of AI model input/output include: input: “user emotion score (stress 0.8, relaxation 0.1, fatigue 0.1), recommended recipe ID: 12345”; output: “recipe description: ‘A simple recipe that just mixes the ingredients and bakes’”; “step list: 1. Put ingredients in a bowl 2. Mix well 3. Bake in a frying pan,” etc. The suggestion unit sends the generated recipe suggestion text to the display unit and presents it to the user terminal in the optimal expression format. These processes, unlike conventional uniform recipe displays or subjective human adjustment of expression, combine automatic emotion estimation by AI and parameter control of suggestion expression to realize personalized information presentation that responds to the user's psychological state. As a technical effect, the suggestion unit can reduce stress caused by information overload or difficulty in understanding and improve the rate of recipe practice and continuity of healthy behavior by optimizing recipe expression according to the user's emotion. Specific application fields include personal health management apps, cooking support services for seniors, corporate welfare apps, and nutritional guidance support in medical institutions.

The suggestion unit is capable of adjusting the level of detail of suggestions when proposing recipes based on the user's physical condition. The suggestion unit, for example, analyzes the user's physical condition using AI. For instance, if the user is tired, the suggestion unit can propose recipes with simple procedures. If the user is in a healthy state, the suggestion unit can propose recipes with detailed procedures. Furthermore, if the user is feeling stressed, the suggestion unit can propose recipes using ingredients with relaxing effects. By adjusting the level of detail of suggestions based on the user's physical condition, the suggestion unit enables more appropriate recipe suggestions. Specifically, the suggestion unit inputs user physical condition evaluation data received from the analysis unit (e.g., fatigue score 0.8, stress level 0.6, health score 0.9) as an input tensor to the recipe suggestion AI model. The AI model dynamically controls suggestion detail parameters (e.g., number of steps, length of explanation, number of divided cooking processes) according to the physical condition scores. Examples of inputs to the AI model include “fatigue score 0.8,” “stress level 0.6,” “health score 0.9,” etc. Examples of outputs from the AI model include “suggestion detail level (simple, standard, detailed),” “recommended recipe ID,” “recipe description,” etc. For example, if the fatigue score is high, recipes with three steps or less and cooking times within 10 minutes are prioritized, and the explanation is generated concisely in short sentences. If the health score is high, detailed recipes with five or more steps and explanations including cooking tips and nutritional commentary are generated. If the stress level is high, recipes containing ingredients expected to have relaxing effects (e.g., herbs, seafood, dairy products) are prioritized, and supplementary information such as “relaxing effects can be expected” is automatically added to the explanation. Examples of AI model input/output include: input: “fatigue 0.9, health 0.3, stress 0.2”; output: “suggestion detail: simple, recipe description: ‘A simple salad that can be made in 3 minutes,’” etc. The suggestion unit sends the generated recipe suggestion text to the display unit and presents it to the user terminal at the optimal level of detail. These processes, unlike conventional uniform recipe displays or subjective human adjustment of detail level, combine automatic analysis of physical condition by AI and parameter control of suggestion detail to realize personalized information presentation that responds to changes in the user's state. As a technical effect, the suggestion unit can reduce stress caused by information overload or difficulty in understanding and improve the rate of recipe practice and continuity of healthy behavior by optimizing the level of detail of recipe suggestions according to the user's physical condition. Specific application fields include personal health management apps, cooking support services for seniors, corporate welfare apps, and nutritional guidance support in medical institutions.

The suggestion unit is capable of applying different suggestion algorithms when proposing recipes according to the user's meal history. The suggestion unit, for example, analyzes the user's meal history using AI. For instance, the suggestion unit can propose recipes similar to meals the user has eaten in the past. The suggestion unit can also consider ingredients the user has avoided in the past and propose recipes that do not include those ingredients. Furthermore, the suggestion unit can propose recipes that consider nutritional balance based on the user's meal history. By applying different suggestion algorithms according to the user's meal history, the suggestion unit enables more appropriate recipe suggestions. Specifically, the suggestion unit obtains meal history data recorded in chronological order for each user (e.g., meal time, ingredient list, calorie intake, nutrient distribution, allergen information) from a structured database and inputs it as an input tensor to the recipe recommendation AI model. The AI model combines multiple recommendation algorithms (e.g., collaborative filtering, content-based filtering, reinforcement learning-based recommendation) to comprehensively analyze the user's meal history patterns, preferences, allergy information, and nutritional balance. Examples of inputs to the AI model include “array of meal history for the past 30 days,” “list of ingredients to avoid,” “nutrient intake history,” etc. Examples of outputs from the AI model include “recommended recipe list,” “fit score for each recipe,” “excluded ingredient list,” etc. For example, recipes similar to dishes frequently eaten in the past are prioritized, and recipes containing ingredients the user has avoided (e.g., allergens or disliked ingredients) are automatically excluded. If nutritional balance is skewed, recipes that supplement deficient nutrients are preferentially proposed. Examples of AI model input/output include: input: “meal history for the past 30 days, ingredients to avoid: eggs, nutrient intake history: vitamin C deficiency”; output: “recommended recipe: ‘Salmon and vegetable foil bake,’ fit score 0.92, excluded ingredient: eggs,” etc. The suggestion unit automatically selects the optimal recommendation algorithm according to the user's meal history and enhances the degree of personalization of recipe suggestions. These processes, unlike conventional simple history referencing or human experience-based suggestions, combine high-dimensional data analysis by AI and dynamic application of multiple algorithms to realize recipe recommendations that respond to each user's preferences, health status, and nutritional balance. As a technical effect, the suggestion unit can greatly improve the accuracy and satisfaction of recipe suggestions by applying diverse recommendation algorithms based on the user's meal history. Specific application fields include personal health management apps, recipe support for people with dietary restrictions, corporate welfare apps, and nutritional guidance support in medical institutions.

The suggestion unit is capable of estimating the user's emotion and adjusting the length of recipe suggestions based on the estimated emotion. The suggestion unit, for example, estimates the user's emotion using an emotion recognition algorithm. For instance, the suggestion unit can analyze the user's facial expressions and voice data to estimate emotion. Furthermore, the suggestion unit can adjust the length of recipe suggestions based on the estimated emotion. For example, if the user is feeling stressed, the suggestion unit proposes short and concise recipes. If the user is relaxed, the suggestion unit can propose recipes with detailed explanations. Additionally, if the user is tired, the suggestion unit can propose recipes that can be prepared quickly. By adjusting the length of recipe suggestions based on the user's emotion, the suggestion unit enables more appropriate recipe suggestions. Emotion estimation is realized, for example, by using an emotion engine or generative AI with emotion estimation functionality. Generative AI may include text generation AI (e.g., LLM) or multimodal generative AI, but is not limited to these examples. Specifically, the suggestion unit simultaneously acquires the user's facial image (e.g., 128×128 pixel RGB image), voice data (e.g., 1-minute audio sampled at 16 kHz), and text data (e.g., diary entries, SNS posts), and inputs these into a multimodal AI model. The emotion estimation AI model adopts an architecture that combines image feature extraction by CNN and emotion analysis of voice and text by RNN or Transformer. Examples of inputs to the AI model include “smiling face image,” “angry face image,” “calm voice,” “high-pitched voice,” “positive text,” and “negative text.” Examples of outputs from the AI model include “emotion labels (stress, relaxation, fatigue)” and “emotion scores (stress 0.8, relaxation 0.1, fatigue 0.1).” Based on the output of the emotion estimation AI model, the suggestion unit dynamically sets the length parameter of the explanation text (e.g., short, standard, detailed) for the recipe suggestion generation AI (e.g., large language model). For example, if the stress score is high, a short recipe with three steps or less is generated; if the relaxation score is high, a detailed explanation including cooking tips and nutritional commentary is generated; if the fatigue score is high, recipes with short cooking times and few steps are prioritized, and catchphrases such as “can be made in 5 minutes” are automatically added. Examples of AI model input/output include: input: “emotion score (stress 0.9, relaxation 0.05, fatigue 0.05), recommended recipe ID: 67890”; output: “recipe description: ‘A simple recipe that just mixes the ingredients and bakes’”; “step list: 1. Put ingredients in a bowl 2. Mix well 3. Bake in a frying pan,” etc. The suggestion unit sends the generated recipe suggestion text to the display unit and presents it to the user terminal at the optimal length. These processes, unlike conventional uniform recipe displays or subjective human adjustment of length, combine automatic emotion analysis by AI and parameter control of suggestion length to realize personalized information presentation that responds to the user's psychological state. As a technical effect, the suggestion unit can reduce stress caused by information overload or difficulty in understanding and improve the rate of recipe practice and continuity of healthy behavior by optimizing the length of recipe explanations according to the user's emotion. Specific application fields include personal health management apps, cooking support services for seniors, corporate welfare apps, and nutritional guidance support in medical institutions.

The suggestion unit is capable of determining the priority of suggestions when proposing recipes based on the user's meal history. The suggestion unit, for example, analyzes the user's meal history using AI. For instance, the suggestion unit can prioritize recipes containing ingredients the user has liked in the past. The suggestion unit can also prioritize recipes that do not contain ingredients the user has avoided in the past. Furthermore, the suggestion unit can prioritize recipes that consider nutritional balance based on the user's meal history. By determining the priority of suggestions based on the user's meal history, the suggestion unit enables more appropriate recipe suggestions. Specifically, the suggestion unit obtains meal history data recorded in chronological order for each user (e.g., meal time, ingredient list, calorie intake, nutrient distribution, allergen information) from a structured database and inputs it as an input tensor (three-dimensional tensor: user×date×number of features) to the recipe recommendation AI model. The AI model combines multiple recommendation algorithms such as content-based filtering, collaborative filtering, and reinforcement learning-based recommendation to comprehensively analyze the user's meal history patterns, preferences, allergy information, and nutritional balance. Examples of inputs to the AI model include “array of meal history for the past 30 days,” “list of ingredients to avoid (e.g., eggs, dairy products),” “nutrient intake history (e.g., vitamin C deficiency),” etc. Examples of outputs from the AI model include “recommended recipe list (e.g., salmon and vegetable foil bake, tofu salad),” “fit score for each recipe (e.g., 0.92, 0.85),” “excluded ingredient list,” etc. Based on the output of the AI model, the suggestion unit places recipes containing ingredients the user has liked in the past at the top of the priority list and automatically excludes recipes containing ingredients the user has avoided. Furthermore, if nutritional balance is skewed, recipes that supplement deficient nutrients are preferentially proposed. As a subsequent process, the suggestion unit sends the recommended recipe list to the display unit and presents it to the user terminal in ranking format or with tags. These processes, unlike conventional simple history referencing or human experience-based suggestions, combine high-dimensional data analysis by AI and dynamic application of multiple algorithms to realize recipe recommendations that respond to each user's preferences, health status, and nutritional balance. As a technical effect, the suggestion unit can greatly improve the accuracy and satisfaction of recipe suggestions by applying diverse recommendation algorithms based on the user's meal history. Specific application fields include personal health management apps, recipe support for people with dietary restrictions, corporate welfare apps, and nutritional guidance support in medical institutions.

The suggestion unit is capable of adjusting the order of suggestions when proposing recipes based on relevance to the user. The suggestion unit, for example, analyzes relevance to the user using AI. For instance, the suggestion unit can propose first recipes containing ingredients the user has liked in the past. The suggestion unit can also propose first recipes that do not contain ingredients the user has avoided in the past. Furthermore, the suggestion unit can propose first recipes that consider nutritional balance based on the user's meal history. By adjusting the order of suggestions based on relevance to the user, the suggestion unit enables more appropriate recipe suggestions. Specifically, the suggestion unit obtains meal history data recorded for each user (e.g., meal time, ingredient list, calorie intake, nutrient distribution, allergen information) from a structured database and inputs it as an input tensor to the recipe recommendation AI model. The AI model combines multiple algorithms such as content-based filtering, collaborative filtering, and reinforcement learning-based recommendation to analyze the user's preferences, past selection tendencies, and nutritional balance. Examples of inputs to the AI model include “meal history for the past 30 days,” “list of ingredients to avoid,” “nutrient intake history,” etc. Examples of outputs from the AI model include “recommended recipe list,” “relevance score for each recipe,” “excluded ingredient list,” etc. Based on the output of the AI model, the suggestion unit places recipes with high relevance scores at the top of the list and places recipes containing ingredients the user has avoided at the bottom or excludes them. If nutritional balance is insufficient, recipes that balance relevance and health effects are preferentially proposed. As a subsequent process, the suggestion unit sends the recommended recipe list to the display unit and presents it to the user terminal in ranking format or with tags. These processes, unlike conventional simple history referencing or human experience-based determination of suggestion order, combine high-dimensional data analysis by AI and dynamic application of multiple algorithms to realize recipe suggestion order that responds to each user's preferences, health status, and nutritional balance. As a technical effect, the suggestion unit can greatly improve satisfaction and practice rate of recipe suggestions by optimizing suggestion order based on relevance to the user. Specific application fields include personal health management apps, recipe support for people with dietary restrictions, corporate welfare apps, and nutritional guidance support in medical institutions.

The display unit is capable of estimating the user's emotion and adjusting the display method of the price list based on the estimated emotion. The display unit, for example, estimates the user's emotion using an emotion recognition algorithm. For instance, the display unit can analyze the user's facial expressions and voice data to estimate emotion. Furthermore, the display unit can adjust the display method of the price list based on the estimated emotion. For example, if the user is feeling stressed, the display unit provides a simple and highly visible display method. If the user is relaxed, the display unit can provide a display method including detailed information. Additionally, if the user is tired, the display unit can provide a concise and easy-to-understand display method. By adjusting the display method of the price list based on the user's emotion, the display unit enables more appropriate display. Emotion estimation is realized, for example, by using an emotion engine or generative AI with emotion estimation functionality. Generative AI may include text generation AI (e.g., LLM) or multimodal generative AI, but is not limited to these examples. Specifically, the display unit simultaneously acquires the user's facial image (e.g., 128×128 pixel RGB image), voice data (e.g., 1-minute audio sampled at 16 kHz), and text data (e.g., SNS posts or diary entries), and inputs these into a multimodal AI model. The emotion estimation AI model adopts an architecture that combines image feature extraction by CNN and emotion analysis of voice and text by RNN or Transformer. Examples of inputs to the AI model include “smiling face image,” “angry face image,” “calm voice,” “high-pitched voice,” “positive text,” and “negative text.” Examples of outputs from the AI model include “emotion labels (stress, relaxation, fatigue)” and “emotion scores (stress 0.8, relaxation 0.1, fatigue 0.1).” Based on the output of the emotion estimation AI model, the display interface control module dynamically sets display method parameters (e.g., amount of information, color scheme, font size, presence of graph display). For example, if the stress score is high, the price list is limited to a list format with large text and simple colors, and detailed information or graph display is omitted. If the relaxation score is high, a rich display mode including price trend graphs and detailed information for each store is provided. If the fatigue score is high, only minimal information is emphasized with large icons or color coding. These controls, unlike conventional uniform displays or subjective human adjustment of display, combine automatic emotion estimation by AI and parameter control of display to realize personalized information presentation that responds to the user's psychological state. As a technical effect, the display unit can reduce stress caused by information overload or difficulty in understanding and realize efficient price comparison and purchase decision-making by optimizing display according to the user's emotion. Specific application fields include personal shopping support apps, purchasing support for seniors, corporate purchasing management systems, and consumer support services for local governments.

The display unit is capable of selecting a display method by referring to the user's past purchase history when displaying the price list. The display unit, for example, analyzes the user's past purchase history using AI. For instance, the display unit can prioritize displaying products the user has purchased in the past. The display unit can also display price fluctuations of products the user has purchased in the past. Furthermore, the display unit can display prices of related products based on the user's purchase history. By referring to the user's past purchase history, the display unit can select the optimal display method. Specifically, the display unit obtains purchase history data recorded for each user (e.g., product ID, product name, purchase date, purchase price, category) from a structured database and inputs it as an input tensor (three-dimensional tensor: user×product×number of features) to the AI analysis model. The AI model analyzes the user's purchasing tendencies and products of interest using content-based filtering and collaborative filtering algorithms. Examples of inputs to the AI model include “list of purchased products in the past year,” “purchase frequency,” “purchase tendency by category,” etc. Examples of outputs from the AI model include “priority display product list,” “price fluctuation graph display flag,” “related product list,” etc. Based on the output of the AI model, the display unit displays products the user has purchased in the past at the top of the list and adds price fluctuation graphs and information on past lowest and highest prices. Furthermore, prices of related products (e.g., new products or similar products in the same category) are displayed simultaneously to support the user's purchase decision. These processes, unlike conventional simple list displays or subjective human selection of display, combine purchase history analysis by AI and parameter control of display to realize personalized information presentation that responds to the user's purchasing tendencies and interests. As a technical effect, the display unit can realize efficient information acquisition, improved accuracy of price comparison, and increased purchase satisfaction by optimizing display based on the user's past purchase history. Specific application fields include personal shopping support apps, corporate purchasing management systems, price survey tools for the distribution industry, and consumer support services for local governments.

The display unit is capable of customizing the display content based on the user's current living situation when displaying the price list. The display unit, for example, analyzes the user's current living situation using AI. For instance, if the user is on a diet, the display unit can prioritize displaying prices of low-calorie foods. If the user is about to undergo a health checkup, the display unit can prioritize displaying prices of health foods. Furthermore, if the user is searching for a specific ingredient, the display unit can prioritize displaying the price of that ingredient. By customizing the display content based on the user's current living situation, the display unit enables more appropriate display. Specifically, the display unit integrates the user's profile information (e.g., age, gender, medical history), recent lifelog data (e.g., weight changes, scheduled health checkup date), and interests set in the application (e.g., diet, muscle building, blood pressure management) and inputs these as feature vectors to the AI model. The AI model uses content-based filtering and reinforcement learning-based filtering models to automatically select product categories and ingredients most relevant to the user's current goals and interests. Examples of inputs to the AI model include “diet goal set,” “two weeks until health checkup,” “searching for specific ingredient,” etc. Examples of outputs from the AI model include “priority display product categories (e.g., low-calorie foods, health foods, specified ingredients),” “display priority score,” etc. Based on the output of the AI model, the display unit presents price information for low-calorie foods, health foods, and specified ingredients at the top of the list or with emphasis, and other products are displayed lower or hidden. These processes, unlike conventional uniform displays or subjective human customization of display, combine living situation analysis by AI and parameter control of display to realize personalized information presentation that responds to the user's goals and interests. As a technical effect, the display unit can realize efficient information acquisition, rapid purchase decision-making, and improved continuity of healthy behavior by optimizing display content according to the user's current living situation. Specific application fields include personalized health management apps, diet support services, fitness monitoring, and lifestyle guidance support in medical institutions.

The display unit is capable of estimating the user's emotion and determining the priority of the price list based on the estimated emotion. The display unit, for example, estimates the user's emotion using an emotion recognition algorithm. For instance, the display unit can analyze the user's facial expressions and voice data to estimate emotion. Furthermore, the display unit can determine the priority of the price list based on the estimated emotion. For example, if the user is feeling stressed, the display unit prioritizes displaying prices of products that help reduce stress. If the user is relaxed, the display unit can prioritize displaying prices of products with relaxing effects. Additionally, if the user is tired, the display unit can prioritize displaying prices of products that help recover from fatigue. By determining the priority of the price list based on the user's emotion, the display unit enables more appropriate display. Emotion estimation is realized, for example, by using an emotion engine or generative AI with emotion estimation functionality. Generative AI may include text generation AI (e.g., LLM) or multimodal generative AI, but is not limited to these examples. Specifically, the display unit simultaneously acquires the user's facial image (e.g., 128×128 pixel RGB image), voice data (e.g., 1-minute audio sampled at 16 kHz), and text data (e.g., SNS posts or diary entries), and inputs these into a multimodal AI model. The emotion estimation AI model adopts an architecture that combines image feature extraction by CNN and emotion analysis of voice and text by RNN or Transformer. Examples of inputs to the AI model include “smiling face image,” “angry face image,” “calm voice,” “high-pitched voice,” “positive text,” and “negative text.” Examples of outputs from the AI model include “emotion labels (stress, relaxation, fatigue)” and “emotion scores (stress 0.8, relaxation 0.1, fatigue 0.1).” Based on the output of the emotion estimation AI model, the display unit calculates priority scores for each product category and displays stress-relief products (e.g., herbal tea, aroma goods), relaxation-effect products (e.g., bath additives, relaxation foods), and fatigue-recovery products (e.g., vitamin drinks, protein bars) at the top of the list. These controls, unlike conventional uniform displays or subjective human determination of priority, combine automatic emotion estimation by AI and parameter control of display to realize personalized information presentation that responds to the user's psychological state. As a technical effect, the display unit can realize rapid purchase decision-making, improved continuity of healthy behavior, and increased user satisfaction by optimizing the priority of the price list according to the user's emotion. Specific application fields include personal shopping support apps, stress management support, corporate welfare apps, and health guidance support in medical institutions.

The display unit is capable of selecting a display method by considering the user's geographic location information when displaying the price list. The display unit, for example, identifies the user's geographic location information using GPS data. For instance, if the user is traveling, the display unit can prioritize displaying prices of stores around the current location. If the user is at home, the display unit can prioritize displaying prices of nearby stores. Furthermore, if the user is at the workplace, the display unit can prioritize displaying prices of stores around the workplace. By considering the user's geographic location information and selecting the optimal display method, the display unit enables more appropriate display. Specifically, the display unit acquires the user's current location (latitude, longitude, altitude, timestamp) with high accuracy using GPS sensors or smartphone location APIs and collaborates with a geographic information database to identify the user's living area and movement history. The AI model receives the current location, movement patterns, and facility usage history as input vectors and outputs the priority of relevant stores and product categories. Examples of inputs to the AI model include “current location: tourist spot,” “at home,” “around workplace,” etc. Examples of outputs from the AI model include “priority display store list,” “display priority score,” etc. Based on the output of the AI model, the display unit displays prices of stores around tourist spots during travel, prices of nearby stores when at home, and prices of stores around the workplace when at work at the top of the list. These processes, unlike conventional uniform displays or subjective human selection of display, combine geographic information analysis by AI and parameter control of display to realize personalized information presentation that responds to the user's behavioral situation and environment. As a technical effect, the display unit can realize efficient information acquisition, rapid purchase decision-making, and increased user satisfaction by optimizing display content according to the user's geographic location information. Specific application fields include personal shopping support apps, purchasing support for travelers, corporate purchasing management systems, and consumer support services for local governments.

The display unit is capable of proposing display content by analyzing the user's social media activity when displaying the price list. The display unit, for example, analyzes the user's social media activity using AI. For instance, the display unit can prioritize displaying prices of products that are trending in the user's social media posts. The display unit can also display prices of products recommended by influencers followed by the user. Furthermore, the display unit can prioritize displaying prices of products the user has shown interest in. By analyzing the user's social media activity, the display unit can propose more appropriate display content. Specifically, the display unit automatically acquires public post data (e.g., text, images, videos, posting time, location tags) linked to the user ID from multiple social media platforms used by the user (e.g., microblogs, photo sharing services, video posting sites) via API or web scraping technology. The display unit inputs the acquired post data into natural language processing AI (e.g., large language model, BERT-based model) and image recognition AI (e.g., CNN-based image classification model) to perform category classification of post content (e.g., product name, category, trendiness) and emotion analysis (e.g., positive, negative, neutral). Examples of inputs to the AI model include “post text about trending products,” “influencer-recommended product names,” “images of products of interest,” etc. Examples of outputs from the AI model include “priority display product list,” “trendiness score,” “related product list,” etc. Based on the output of the AI model, the display unit displays products with high trendiness scores, influencer-recommended products, and products the user has shown interest in at the top of the price list. These processes, unlike conventional uniform displays or subjective human selection of display, combine social media activity analysis by AI and parameter control of display to realize personalized information presentation that responds to the user's interests and trends. As a technical effect, the display unit can realize efficient information acquisition, rapid purchase decision-making, and increased user satisfaction by optimizing display content based on the user's social media activity. Specific application fields include personalized shopping support apps, trend product recommendation services, corporate marketing support, and consumer support services for local governments.

The system according to the embodiment is not limited to the examples described above and can be variously modified as follows. Specifically, the system can be applied to a wide range of fields, not limited to health management, such as fitness monitoring, meal management, stress management, sleep improvement, lifestyle disease prevention, corporate employee health support, remote health observation in medical institutions, and health promotion services by local governments. In addition, the architecture of the AI model may adopt various configurations such as convolutional neural networks, recurrent neural networks, Transformer-based models, graph neural networks, self-supervised learning models, and multimodal integration models. Data collection means may also utilize various hardware and software configurations such as wearable devices, smartphones, IoT sensors, cloud-linked databases, and external APIs. Furthermore, the user interface can be extended to various forms such as smartphone apps, web apps, voice-interactive interfaces, smart watch integration, and AR/VR displays. These variations allow the system to be flexibly customized according to user needs and usage environments, and the technical effects include improved comprehensiveness and accuracy of data acquisition, enhanced personalization of analysis and recommendations, optimization of user experience, efficient computation and communication load, and improved accuracy of health risk detection and behavior change support. Specific application fields include personal health management apps, corporate health management support, diagnostic support in medical institutions, health promotion services by local governments, fitness gym member management, and health incentive programs by insurance companies.

The analysis unit is capable of estimating the user's emotion and adjusting the criteria for physical condition evaluation based on the estimated emotion. For example, if the user is feeling stressed, the analysis unit can perform physical condition evaluation with emphasis on stress level. If the user is relaxed, the analysis unit can perform physical condition evaluation with emphasis on overall health status. Furthermore, if the user is tired, the analysis unit can perform physical condition evaluation with emphasis on fatigue level. By adjusting the criteria for physical condition evaluation based on the user's emotion, the analysis unit enables more accurate physical condition evaluation. Specifically, the analysis unit simultaneously acquires the user's facial image (e.g., 128×128 pixel RGB image), voice data (e.g., 1-minute audio sampled at 16 kHz), and text data (e.g., diary entries, SNS posts), and inputs these into a multimodal AI model. The emotion estimation AI model adopts an architecture that combines image feature extraction by CNN and emotion analysis of voice and text by RNN or Transformer. Examples of inputs to the AI model include “smiling face image,” “angry face image,” “calm voice,” “high-pitched voice,” “positive text,” and “negative text.” Examples of outputs from the AI model include “emotion labels (stress, relaxation, fatigue)” and “emotion scores (stress 0.8, relaxation 0.1, fatigue 0.1).” Based on the output of the emotion estimation AI model, the analysis unit dynamically adjusts the weight parameters and evaluation criteria of the physical condition evaluation algorithm. For example, if the stress score is high, the physical condition evaluation model gives weight to stress-related indicators (e.g., heart rate variability, sleep quality, cortisol level); if the relaxation score is high, overall health indicators (e.g., nutritional balance, amount of exercise, sleep duration) are emphasized; if the fatigue score is high, fatigue evaluation indicators (e.g., subjective fatigue score, muscle pain, rest time) are preferentially evaluated. These adjustments are realized by the analysis unit automatically changing weighting and threshold settings based on AI output values. As a technical effect, the analysis unit can realize highly accurate and personalized physical condition evaluation that responds to changes in the user's state by dynamically optimizing evaluation criteria according to the user's emotional state, compared to conventional uniform evaluation or manual settings. This enables early detection of health risks and appropriate health support, contributing to improved user experience and efficiency in medical and health management. Specific application fields include personal health management apps, stress management support, corporate employee health monitoring, and remote health observation in medical institutions.

The suggestion unit is capable of estimating the user's emotion and adjusting the method of recipe suggestion expression based on the estimated emotion. For example, if the user is feeling stressed, the suggestion unit can propose simple and easy-to-understand recipes. If the user is relaxed, the suggestion unit can propose recipes with detailed procedures. Furthermore, if the user is tired, the suggestion unit can propose recipes that are easy to prepare. By adjusting the method of recipe suggestion expression based on the user's emotion, the suggestion unit enables more appropriate recipe suggestions. Specifically, the suggestion unit simultaneously acquires the user's facial image (e.g., 128×128 pixel RGB image), voice data (e.g., 1-minute audio sampled at 16 kHz), and text data (e.g., diary entries, SNS posts), and inputs these into a multimodal AI model. The emotion estimation AI model adopts an architecture that combines image feature extraction by CNN and emotion analysis of voice and text by RNN or Transformer. Examples of inputs to the AI model include “smiling face image,” “angry face image,” “calm voice,” “high-pitched voice,” “positive text,” and “negative text.” Examples of outputs from the AI model include “emotion labels (stress, relaxation, fatigue, joy, sadness)” and “emotion scores (stress 0.7, relaxation 0.2, fatigue 0.1).” Based on the output of the emotion estimation AI model, the suggestion unit dynamically sets suggestion expression parameters (e.g., level of detail in explanation, conciseness of steps, softness of tone) for the recipe suggestion generation AI (e.g., large language model). For example, if the stress score is high, the recipe explanation is generated as short sentences and mainly bullet points, and the suggestion text is limited to the minimum cooking steps. If the relaxation score is high, a long recipe including detailed explanations such as cooking tips, arrangement examples, and health effects of ingredients is generated. If the fatigue score is high, recipes with short cooking times and few steps are prioritized, and catchphrases such as “can be made in 5 minutes” or “only 3 ingredients” are automatically added. Examples of AI model input/output include: input: “user emotion score (stress 0.8, relaxation 0.1, fatigue 0.1), recommended recipe ID: 12345”; output: “recipe description: ‘A simple recipe that just mixes the ingredients and bakes’”; “step list: 1. Put ingredients in a bowl 2. Mix well 3. Bake in a frying pan,” etc. The suggestion unit sends the generated recipe suggestion text to the display unit and presents it to the user terminal in the optimal expression format. These processes, unlike conventional uniform recipe displays or subjective human adjustment of expression, combine automatic emotion estimation by AI and parameter control of suggestion expression to realize personalized information presentation that responds to the user's psychological state. As a technical effect, the suggestion unit can reduce stress caused by information overload or difficulty in understanding and improve the rate of recipe practice and continuity of healthy behavior by optimizing recipe expression according to the user's emotion. Specific application fields include personal health management apps, cooking support services for seniors, corporate welfare apps, and nutritional guidance support in medical institutions.

The display unit is capable of estimating the user's emotion and adjusting the display method of the price list based on the estimated emotion. For example, if the user is feeling stressed, the display unit can provide a simple and highly visible display method. If the user is relaxed, the display unit can provide a display method including detailed information. Furthermore, if the user is tired, the display unit can provide a concise and easy-to-understand display method. By adjusting the display method of the price list based on the user's emotion, the display unit enables more appropriate display. Specifically, the display unit simultaneously acquires the user's facial image (e.g., 128×128 pixel RGB image), voice data (e.g., 1-minute audio sampled at 16 kHz), and text data (e.g., SNS posts or diary entries), and inputs these into a multimodal AI model. The emotion estimation AI model adopts an architecture that combines image feature extraction by CNN and emotion analysis of voice and text by RNN or Transformer. Examples of inputs to the AI model include “smiling face image,” “angry face image,” “calm voice,” “high-pitched voice,” “positive text,” and “negative text.” Examples of outputs from the AI model include “emotion labels (stress, relaxation, fatigue)” and “emotion scores (stress 0.8, relaxation 0.1, fatigue 0.1).” Based on the output of the emotion estimation AI model, the display interface control module dynamically sets display method parameters (e.g., amount of information, color scheme, font size, presence of graph display). For example, if the stress score is high, the price list is limited to a list format with large text and simple colors, and detailed information or graph display is omitted. If the relaxation score is high, a rich display mode including price trend graphs and detailed information for each store is provided. If the fatigue score is high, only minimal information is emphasized with large icons or color coding. These controls, unlike conventional uniform displays or subjective human adjustment of display, combine automatic emotion estimation by AI and parameter control of display to realize personalized information presentation that responds to the user's psychological state. As a technical effect, the display unit can reduce stress caused by information overload or difficulty in understanding and realize efficient price comparison and purchase decision-making by optimizing display according to the user's emotion. Specific application fields include personal shopping support apps, purchasing support for seniors, corporate purchasing management systems, and consumer support services for local governments.

The collection unit is capable of estimating the user's emotion and adjusting the timing of lifelog data collection based on the estimated emotion. For example, if the user is feeling stressed, the collection unit can collect lifelog data during relaxed periods. If the user is relaxed, the collection unit can collect lifelog data during active periods. Furthermore, if the user is tired, the collection unit can prioritize collecting data during rest. By adjusting the timing of lifelog data collection based on the user's emotion, the collection unit enables more accurate data collection. Specifically, the collection unit simultaneously acquires the user's facial image data (e.g., 128×128 pixel RGB face image tensor), voice waveform data (e.g., 1-minute audio sampled at 16 kHz), and text data (e.g., user diary or SNS posts), and inputs these as a multimodal input tensor to the emotion estimation AI model. The emotion estimation AI model adopts a multimodal architecture combining facial expression feature extraction by convolutional neural network (CNN), voice emotion recognition by recurrent neural network (RNN) or Transformer-based models, and text emotion analysis by natural language processing models (e.g., BERT-based). Examples of inputs to the AI model include “smiling face image,” “angry face image,” “calm voice waveform,” “high-pitched fast voice waveform,” “positive diary text,” “negative SNS post,” etc. Examples of outputs from the AI model include “emotion labels (e.g., stress, relaxation, fatigue, joy, sadness)” and “emotion scores (e.g., stress 0.7, relaxation 0.2, fatigue 0.1).” Based on the output of the emotion estimation AI model, the collection unit controls the timing of lifelog data collection (e.g., heart rate, sleep duration, amount of exercise, meal contents, blood pressure). For example, if the stress score is high, data collection is concentrated during nighttime or holidays when the user is estimated to be relaxed. If the relaxation score is high, data collection is performed during active periods (e.g., morning or after exercise). If the fatigue score is high, biometric data during rest or sleep (e.g., sleep depth, heart rate variability) is prioritized for collection. These controls are realized by the scheduler module of the collection unit automatically adjusting collection timing based on AI output values. As a technical effect, the collection unit can realize highly accurate and efficient data acquisition that responds to changes in the user's state by dynamically optimizing collection timing according to the user's emotional state, compared to conventional fixed-time or uniform collection. This improves the accuracy of physical condition estimation and health risk detection and reduces unnecessary data collection and computational/communication load. Specific application fields include personal health management applications, stress management support systems, corporate employee mental health monitoring, and remote health observation in medical institutions.

The suggestion unit is capable of estimating the user's emotion and adjusting the length of recipe suggestions based on the estimated emotion. For example, if the user is feeling stressed, the suggestion unit can propose short and concise recipes. If the user is relaxed, the suggestion unit can propose recipes with detailed explanations. Furthermore, if the user is tired, the suggestion unit can propose recipes that can be prepared quickly. By adjusting the length of recipe suggestions based on the user's emotion, the suggestion unit enables more appropriate recipe suggestions. Specifically, the suggestion unit simultaneously acquires the user's facial image (e.g., 128×128 pixel RGB image), voice data (e.g., 1-minute audio sampled at 16 kHz), and text data (e.g., diary entries, SNS posts), and inputs these into a multimodal AI model. The emotion estimation AI model adopts an architecture that combines image feature extraction by CNN and emotion analysis of voice and text by RNN or Transformer. Examples of inputs to the AI model include “smiling face image,” “angry face image,” “calm voice,” “high-pitched voice,” “positive text,” and “negative text.” Examples of outputs from the AI model include “emotion labels (stress, relaxation, fatigue)” and “emotion scores (stress 0.8, relaxation 0.1, fatigue 0.1).” Based on the output of the emotion estimation AI model, the suggestion unit dynamically sets the length parameter of the explanation text (e.g., short, standard, detailed) for the recipe suggestion generation AI (e.g., large language model). For example, if the stress score is high, a short recipe with three steps or less is generated; if the relaxation score is high, a detailed explanation including cooking tips and nutritional commentary is generated; if the fatigue score is high, recipes with short cooking times and few steps are prioritized, and catchphrases such as “can be made in 5 minutes” are automatically added. Examples of AI model input/output include: input: “emotion score (stress 0.9, relaxation 0.05, fatigue 0.05), recommended recipe ID: 67890”; output: “recipe description: ‘A simple recipe that just mixes the ingredients and bakes’”; “step list: 1. Put ingredients in a bowl 2. Mix well 3. Bake in a frying pan,” etc. The suggestion unit sends the generated recipe suggestion text to the display unit and presents it to the user terminal at the optimal length. These processes, unlike conventional uniform recipe displays or subjective human adjustment of length, combine automatic emotion analysis by AI and parameter control of suggestion length to realize personalized information presentation that responds to the user's psychological state. As a technical effect, the suggestion unit can reduce stress caused by information overload or difficulty in understanding and improve the rate of recipe practice and continuity of healthy behavior by optimizing the length of recipe explanations according to the user's emotion. Specific application fields include personal health management apps, cooking support services for seniors, corporate welfare apps, and nutritional guidance support in medical institutions.

The collection unit is capable of analyzing the user's past lifelog data and selecting a collection method. For example, the collection unit can analyze the user's past sleep data and determine the optimal collection timing. The collection unit can also analyze exercise data and prioritize data collection after exercise. Furthermore, the collection unit can analyze meal data and collect data after meals. By analyzing the user's past lifelog data, the collection unit can select the optimal collection method. Specifically, the collection unit stores multidimensional arrays such as sleep duration data recorded chronologically for each user (e.g., daily bedtime and wake-up time, total sleep duration), amount of exercise data (e.g., number of steps, calories burned, exercise intensity), and meal content data (e.g., meal time, calorie intake, nutrient distribution) in a structured database keyed by user ID and timestamp. The collection unit inputs these past data as input tensors (e.g., three-dimensional tensor: user×date×number of features) to the AI analysis model. The AI model uses recurrent neural networks (RNN) or Transformer-based time series pattern recognition models to extract each user's lifestyle rhythm and behavioral patterns. Examples of inputs to the AI model include “array of sleep duration for the past 30 days,” “weekly exercise amount vector,” “time series array of meal times,” etc. Examples of outputs from the AI model include “optimal data collection timing (e.g., every day at 22:00, within 30 minutes after exercise, within 1 hour after meals),” “collection frequency (e.g., three times a day, additional collection on exercise days),” etc. Based on the output of the AI model, the collection scheduler module automatically adjusts data collection timing and frequency. For example, if the user has a nocturnal lifestyle, sleep data is intensively collected at night; if the user has an exercise habit, biometric data (e.g., heart rate, blood pressure) is preferentially collected immediately after exercise; if the meal pattern is irregular, data collection is performed in accordance with the input timing of the meal recording app. As a technical effect, the collection unit can greatly improve the comprehensiveness, usefulness, and reliability of data by automatically generating personalized collection strategies based on each user's past data, compared to conventional uniform collection or manual settings. In addition, unnecessary data collection can be suppressed, and optimization of computational and communication resources can be achieved. Specific application fields include personal health management apps, lifestyle disease prevention programs, corporate health management support, and remote monitoring in medical institutions.

The display unit is capable of selecting a display method by referring to the user's past purchase history when displaying the price list. For example, the display unit can prioritize displaying products the user has purchased in the past. The display unit can also display price fluctuations of products the user has purchased in the past. Furthermore, the display unit can display prices of related products based on the user's purchase history. By referring to the user's past purchase history, the display unit can select the optimal display method. Specifically, the display unit obtains purchase history data recorded for each user (e.g., product ID, product name, purchase date, purchase price, category) from a structured database and inputs it as an input tensor (three-dimensional tensor: user×product×number of features) to the AI analysis model. The AI model analyzes the user's purchasing tendencies and products of interest using content-based filtering and collaborative filtering algorithms. Examples of inputs to the AI model include “list of purchased products in the past year,” “purchase frequency,” “purchase tendency by category,” etc. Examples of outputs from the AI model include “priority display product list,” “price fluctuation graph display flag,” “related product list,” etc. Based on the output of the AI model, the display unit displays products the user has purchased in the past at the top of the list and adds price fluctuation graphs and information on past lowest and highest prices. Furthermore, prices of related products (e.g., new products or similar products in the same category) are displayed simultaneously to support the user's purchase decision. These processes, unlike conventional simple list displays or subjective human selection of display, combine purchase history analysis by AI and parameter control of display to realize personalized information presentation that responds to the user's purchasing tendencies and interests. As a technical effect, the display unit can realize efficient information acquisition, improved accuracy of price comparison, and increased purchase satisfaction by optimizing display based on the user's past purchase history. Specific application fields include personal shopping support apps, corporate purchasing management systems, price survey tools for the distribution industry, and consumer support services for local governments.

The suggestion unit can apply different suggestion algorithms according to the user's meal history when proposing recipes. For example, it can propose recipes similar to those the user has eaten in the past. It can also consider ingredients the user has avoided in the past and propose recipes that do not include those ingredients. Furthermore, it can propose recipes that take nutritional balance into account based on the user's meal history. By applying different suggestion algorithms according to the user's meal history, the suggestion unit can provide more appropriate recipe suggestions. Specifically, the suggestion unit acquires meal history data recorded in chronological order for each user (e.g., meal time, ingredient list, calorie intake, nutrient distribution, allergen information) from a structured database and inputs this as an input tensor to a recipe recommendation AI model. The AI model combines multiple recommendation algorithms (e.g., collaborative filtering, content-based filtering, reinforcement learning-based recommendation) to comprehensively analyze the user's meal history patterns, preferences, allergy information, and nutritional balance. Examples of inputs to the AI model include “meal history array for the past 30 days,” “list of ingredients to avoid,” and “nutrient intake history.” Examples of outputs from the AI model include “recommended recipe list,” “fitness score for each recipe,” and “excluded ingredient list.” For example, recipes similar to dishes frequently eaten in the past are preferentially suggested, and recipes containing ingredients avoided in the past (e.g., allergens or disliked ingredients) are automatically excluded. If the nutritional balance is biased, recipes that supplement deficient nutrients are preferentially proposed. An example of AI model input/output is: input: “meal history for the past 30 days, ingredients to avoid: eggs, nutrient intake history: vitamin C deficiency”; output: “recommended recipe: ‘Salmon and Vegetable Foil Bake,’ fitness score 0.92, excluded ingredient: eggs.” The suggestion unit automatically selects the optimal recommendation algorithm according to the user's meal history, thereby enhancing the degree of personalization in recipe suggestions. These processes differ from conventional simple history referencing or human heuristics, and by utilizing high-dimensional data analysis and dynamic application of multiple algorithms by AI, recipe recommendations that promptly respond to each user's preferences, health status, and nutritional balance are realized. As a technical effect, the suggestion unit can greatly improve the accuracy and satisfaction of recipe suggestions by applying diverse recommendation algorithms based on the user's meal history. Specific application fields include personal health management apps, recipe support for people with dietary restrictions, corporate welfare apps, and nutritional guidance support for medical institutions.

The collection unit can preferentially collect highly relevant data within a radius of 1 kilometer from the user's current location by considering the user's geographic location information when collecting lifelog data. For example, when the user is traveling, data related to travel distance and number of steps can be preferentially collected. When the user is at home, data related to sleep and meal contents can be preferentially collected. By considering the user's geographic location information and preferentially collecting highly relevant data, the collection unit can collect more appropriate data. Specifically, the collection unit acquires the user's current location (latitude, longitude, altitude, timestamp) with high accuracy using GPS sensors or smartphone location APIs, and collaborates with a geographic information database to identify the user's living area and movement history. The collection unit obtains facility information within a radius of 1 kilometer from the current location, such as stores, restaurants, parks, and gyms, and also collects facility usage history and surrounding environmental data (e.g., temperature, humidity, noise level). The AI model receives the user's current location, movement patterns, and facility usage history as input vectors and outputs the priority of highly relevant lifelog data types (e.g., travel distance, number of steps, meal contents, sleep duration, heart rate). Examples of inputs to the AI model include “current location: tourist spot,” “travel distance: 5 km,” “facility usage: gym,” and “staying at home.” Examples of outputs from the AI model include “priority data types for collection (e.g., travel distance and number of steps while traveling, sleep and meal contents at home)” and “collection priority score.” Based on the output results of the AI model, the collection unit collects travel distance and number of steps more frequently while traveling, and focuses on collecting sleep data and meal contents while staying at home. This enables optimal data collection according to the user's geographic location and behavioral status. As a technical effect, the collection unit achieves high-precision data acquisition that promptly responds to changes in user behavior and environment, compared to conventional uniform collection, by dynamically controlling data collection using geographic location information, thereby improving the accuracy of physical condition estimation and health support, and optimizing computational and communication loads. Specific application fields include personal health management apps, health monitoring for travelers, corporate employee health support, and municipal health promotion services.

The analysis unit can improve the accuracy of physical condition evaluation by considering interrelationships among lifelog data (for example, the relationship between sleep duration and amount of exercise) during analysis. For example, it can analyze the correlation between sleep data and heart rate data and reflect this in physical condition evaluation. It can also analyze the correlation between exercise data and meal data and reflect this in physical condition evaluation. Furthermore, it can analyze the correlation between stress level and sleep data and reflect this in physical condition evaluation. By considering the interrelationships among lifelog data, the analysis unit can improve the accuracy of physical condition evaluation. Specifically, the analysis unit structures multidimensional lifelog data recorded in chronological order for each user (e.g., sleep duration, amount of exercise, meal contents, heart rate, stress score) as a three-dimensional tensor (user×time×number of features) and inputs this to an AI analysis model. The AI analysis model adopts a multitask learning-type neural network (e.g., Transformer with multi-head attention, multi-stream RNN) to automatically extract interrelationships among features (e.g., sleep and exercise, meal and heart rate, stress and sleep) in a high-dimensional space. Examples of inputs to the AI model include “sleep duration array for the past 30 days,” “weekly exercise amount vector,” “categorical encoding of meal contents,” “time series array of heart rate,” and “time series of stress score.” Examples of outputs from the AI model include “physical condition evaluation score (0.0-1.0),” “correlation coefficient matrix (e.g., correlation between sleep and exercise: 0.6, correlation between exercise and meal: 0.4),” and “health risk prediction label.” Based on the output results of the AI model, the analysis unit's physical condition evaluation algorithm weights the interrelationships among features to calculate a comprehensive physical condition score. For example, if the correlation between sleep and exercise is high, both are evaluated simultaneously, and if there is a strong negative correlation between stress and sleep, decreased sleep quality during increased stress is considered a risk factor. These processes differ from conventional single-indicator evaluation or human heuristic judgment, and by automatic analysis of high-dimensional data and unconventional feature integration, the accuracy and reliability of physical condition evaluation are greatly improved. As a technical effect, the analysis unit enables early detection of health risks and improves the accuracy of personalized health support by having AI automatically learn and utilize complex interrelationships among lifelog data. Specific application fields include personal health management apps, lifestyle disease prevention programs, corporate health management support, and remote monitoring for medical institutions.

The following is a brief explanation of the processing flow of Example of the Embodiment. Specifically, the present system is composed of multiple hardware and software modules, such as a collection unit, an analysis unit, a suggestion unit, and a display unit. The collection unit collects multidimensional time-series data such as sleep duration (e.g., 7 hours, 5 hours), amount of exercise (e.g., 10,000 steps per day, 30 minutes of running), meal contents (e.g., eggs and toast for breakfast, salad and chicken for lunch), and heart rate (e.g., 70 bpm at rest, 120 bpm during exercise) obtained from wearable devices and smartphone sensors, with a resolution of one day or one minute. GPS data is acquired as vector data including latitude, longitude, altitude, and timestamp. The analysis unit receives these data as input tensors (e.g., three-dimensional tensors of user×time×number of features) and uses convolutional neural networks (CNN), recurrent neural networks (RNN), or Transformer-based time-series analysis models to output the user's physical condition (e.g., fatigue score 0.8, stress level 0.6, sleep deprivation label 1). Examples of inputs to the AI model include “sleep duration array for the past 7 days,” “daily exercise amount vector,” “categorical encoding of meal contents,” and “time series array of heart rate.” Examples of outputs from the AI model include “fatigue score (0.0-1.0),” “estimated stress level (low, medium, high),” and “recommended nutrients (protein, vitamin C).” The suggestion unit receives the output from the analysis unit, searches for recipes that meet the conditions from the recipe database, and applies recommendation algorithms (e.g., collaborative filtering, content-based filtering, reinforcement learning-based recommendation) to select the optimal recipe. For example, if the fatigue score is high, recipes rich in vitamin B group are prioritized, and if sleep deprivation is detected, menus that are easy to digest are prioritized. Examples of recipe suggestion outputs include “chicken breast and broccoli salad” and “salmon foil bake.” The display unit generates a list of ingredients required for the suggested recipe, obtains price information (e.g., one pack of eggs: 200 yen, 100 g of chicken breast: 120 yen) in real time from a store information database or online API within the living area, and displays it on the user terminal in list or graph format. Furthermore, when going out, the display unit collects menu information from restaurant databases around the current location based on GPS data, analyzes menu contents using natural language processing AI (e.g., large language models), and recommends restaurants suitable for the user's physical condition (e.g., Japanese restaurants with easily digestible food, restaurants offering low-calorie menus). Examples of AI inputs include “text data of restaurant menus,” “user physical condition score,” and “current location information,” and examples of outputs include “recommended restaurant list” and “health suitability score for each restaurant.” These series of processes differ from conventional simple recording, judgment, and recommendation by humans, and are realized by advanced application of computer technology, such as automatic analysis of high-dimensional data, integration of multiple data sources, application of unconventional recommendation algorithms, and real-time linkage of price information, thereby achieving significant improvements in processing speed, accuracy of physical condition evaluation and recipe recommendation, optimization of user experience, and efficiency of communication and computation loads. Specific application fields include personal health management applications, corporate employee health support systems, lifestyle guidance support for medical institutions, and municipal health promotion services.

Step 1: The collection unit collects lifelog data and GPS data. Lifelog data includes sleep duration, amount of exercise, meal contents, and heart rate. The collection unit collects these data using wearable devices and smartphones. GPS data provides user location information and is used to obtain information about stores and restaurants within the living area. Step 2: The analysis unit analyzes the data collected by the collection unit and evaluates the user's physical condition. The analysis unit can analyze the data using AI and evaluate the user's fatigue level and stress level. Step 3: The suggestion unit proposes recipes based on the physical condition evaluated by the analysis unit. The suggestion unit uses AI to propose optimal recipes to the user, such as recipes using ingredients effective for recovery from fatigue. Step 4: The display unit displays a price list of ingredients required for the recipe proposed by the suggestion unit. The display unit collects online price information and can obtain and display price information from stores within the user's living area. Furthermore, it can display the latest price information in cooperation with store databases. Specifically, the present system collects multidimensional data such as sleep duration (e.g., 7.5 hours, 5.2 hours), amount of exercise (e.g., 10,000 steps per day, 350 kcal burned), meal contents (e.g., oatmeal for breakfast, chicken salad for lunch), and heart rate (e.g., 65 bpm at rest, 120 bpm during exercise) for each user in chronological order via wearable devices (e.g., accelerometer, heart rate sensor, blood pressure monitor) or smartphone applications. GPS data is acquired as vector data including latitude, longitude, altitude, and timestamp every minute and is used to identify the user's movement history and living area. The analysis unit preprocesses the data received from the collection unit as, for example, a three-dimensional tensor (user×time×number of features), and uses convolutional neural networks (CNN), recurrent neural networks (RNN), or Transformer-based time-series analysis models to estimate the user's physical condition (e.g., fatigue score 0.7, stress level 0.5, sleep deprivation label 1). Examples of AI model inputs include “sleep duration array for the past 7 days,” “daily exercise amount vector,” “categorical encoding of meal contents,” and “time series array of heart rate,” and examples of outputs include “fatigue score (0.0-1.0),” “estimated stress level (low, medium, high),” and “recommended nutrients (protein, vitamin C).” The suggestion unit receives the output from the analysis unit, searches for recipes that meet the conditions from the recipe database, and applies recommendation algorithms (e.g., collaborative filtering, content-based filtering, reinforcement learning-based recommendation) to select the optimal recipe. For example, if the fatigue score is high, recipes rich in vitamin B group are prioritized, and if sleep deprivation is detected, menus that are easy to digest are prioritized. Examples of recipe suggestion outputs include “chicken breast and broccoli salad” and “salmon foil bake.” The display unit generates a list of ingredients required for the suggested recipe, obtains price information (e.g., one pack of eggs: 200 yen, 100 g of chicken breast: 120 yen) in real time from a store information database or online API within the living area, and displays it on the user terminal in list or graph format. Furthermore, the display unit can cooperate with store databases and APIs to obtain and display the latest price information. These series of processes differ from conventional simple recording, judgment, and recommendation by humans, and are realized by advanced application of computer technology, such as automatic analysis of high-dimensional data, integration of multiple data sources, application of unconventional recommendation algorithms, and real-time linkage of price information, thereby achieving significant improvements in processing speed, accuracy of physical condition evaluation and recipe recommendation, optimization of user experience, and efficiency of communication and computation loads. Specific application fields include personal health management applications, corporate employee health support systems, lifestyle guidance support for medical institutions, and municipal health promotion services.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the above-described collection unit, analysis unit, suggestion unit, and display unit is implemented by at least one of, for example, a smart device 14 and a data processing apparatus 12. For example, the collection unit collects lifelog data and GPS data using a wearable device or smartphone of the smart device 14. The analysis unit is implemented, for example, by a specific processing unit 290 of the data processing apparatus 12, and analyzes the data using AI to evaluate the user's physical condition. The suggestion unit is implemented, for example, by the specific processing unit 290 of the data processing apparatus 12, and proposes an optimal recipe to the user using AI. The display unit is implemented, for example, by a control unit 46A of the smart device 14, and displays a price list of ingredients required for the proposed recipe. The correspondence between each unit and the device or control unit is not limited to the above examples and various modifications are possible.

### Second Embodiment

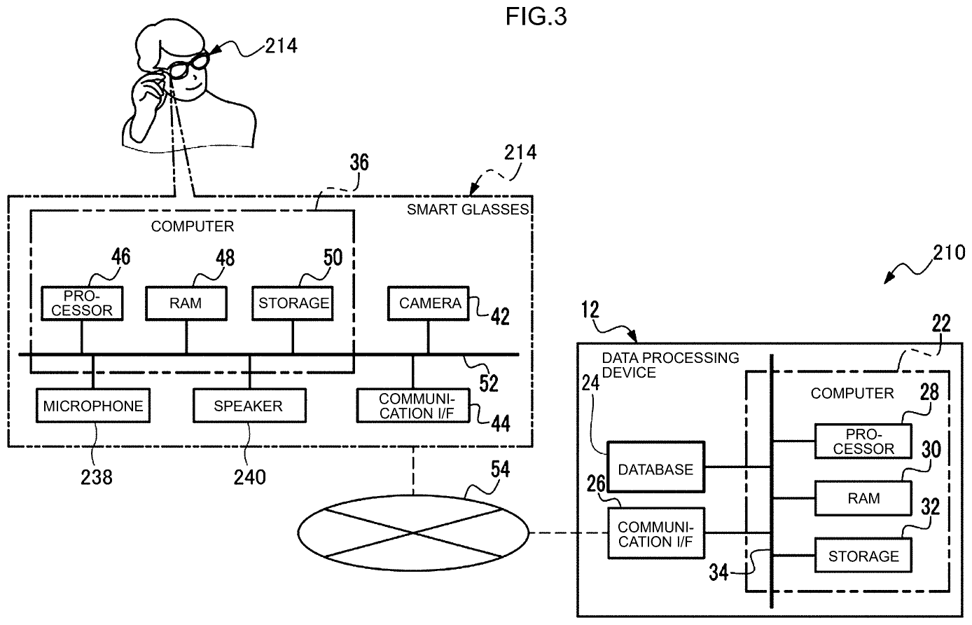

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

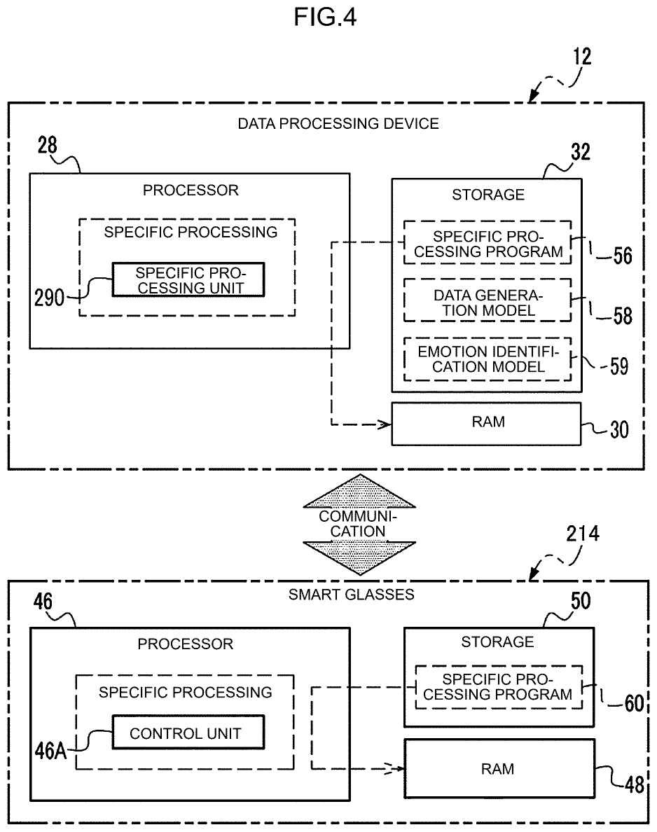

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the above-described collection unit, analysis unit, suggestion unit, and display unit is implemented by at least one of, for example, smart glasses 214 and a data processing apparatus 12. For example, the collection unit collects lifelog data and GPS data using a wearable device or smartphone of the smart glasses 214. The analysis unit is implemented, for example, by a specific processing unit 290 of the data processing apparatus 12, and analyzes the data using AI to evaluate the user's physical condition. The suggestion unit is implemented, for example, by the specific processing unit 290 of the data processing apparatus 12, and proposes an optimal recipe to the user using AI. The display unit is implemented, for example, by a control unit 46A of the smart glasses 214, and displays a price list of ingredients required for the proposed recipe. The correspondence between each unit and the device or control unit is not limited to the above examples and various modifications are possible.

### Third Embodiment

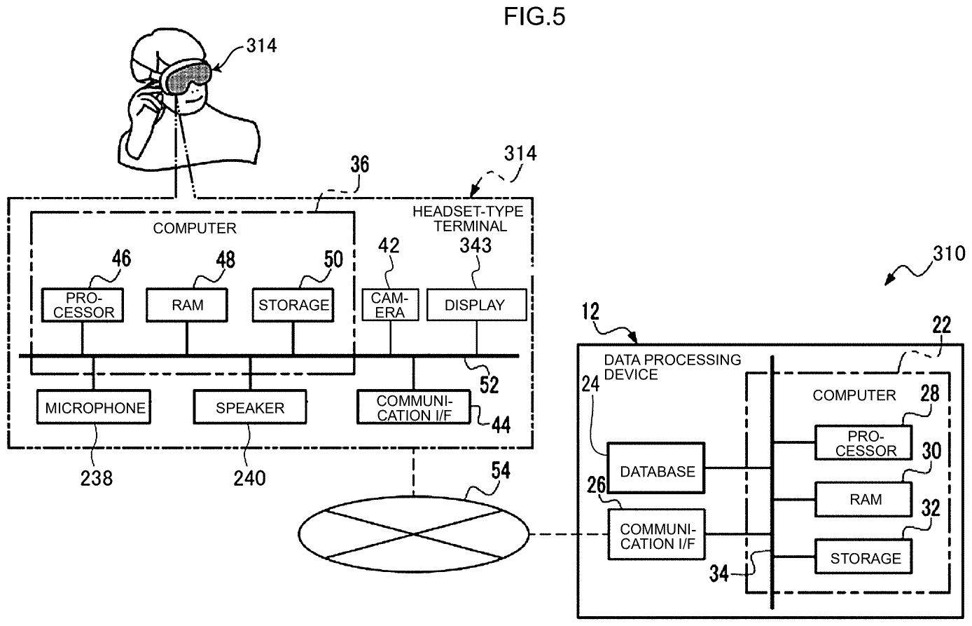

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

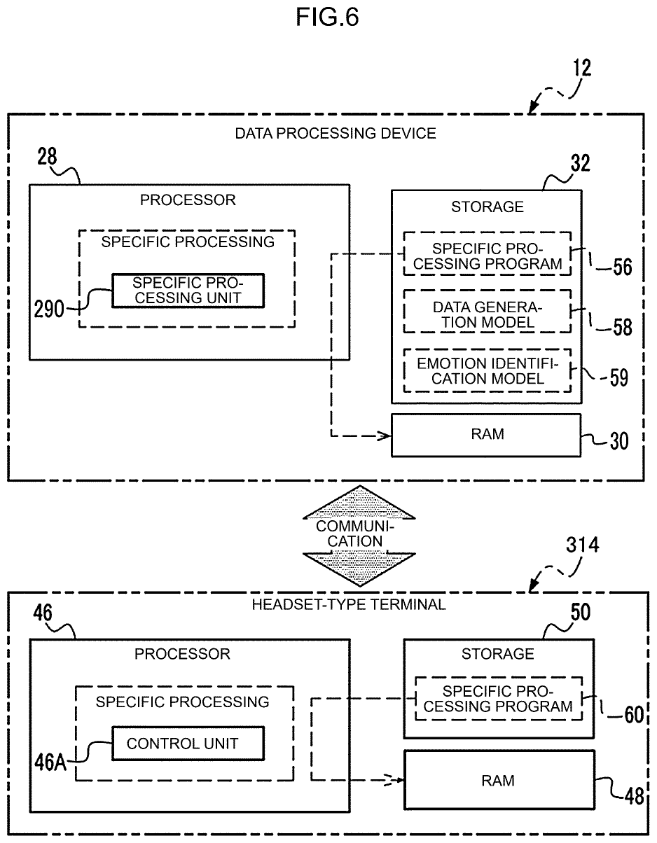

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the above-described collection unit, analysis unit, suggestion unit, and display unit is implemented by at least one of, for example, a headset-type terminal 314 and a data processing apparatus 12. For example, the collection unit collects lifelog data and GPS data using a wearable device or smartphone of the headset-type terminal 314. The analysis unit is implemented, for example, by a specific processing unit 290 of the data processing apparatus 12, and analyzes the data using AI to evaluate the user's physical condition. The suggestion unit is implemented, for example, by the specific processing unit 290 of the data processing apparatus 12, and proposes an optimal recipe to the user using AI. The display unit is implemented, for example, by a control unit 46A of the headset-type terminal 314, and displays a price list of ingredients required for the proposed recipe. The correspondence between each unit and the device or control unit is not limited to the above examples and various modifications are possible.

### Fourth Embodiment

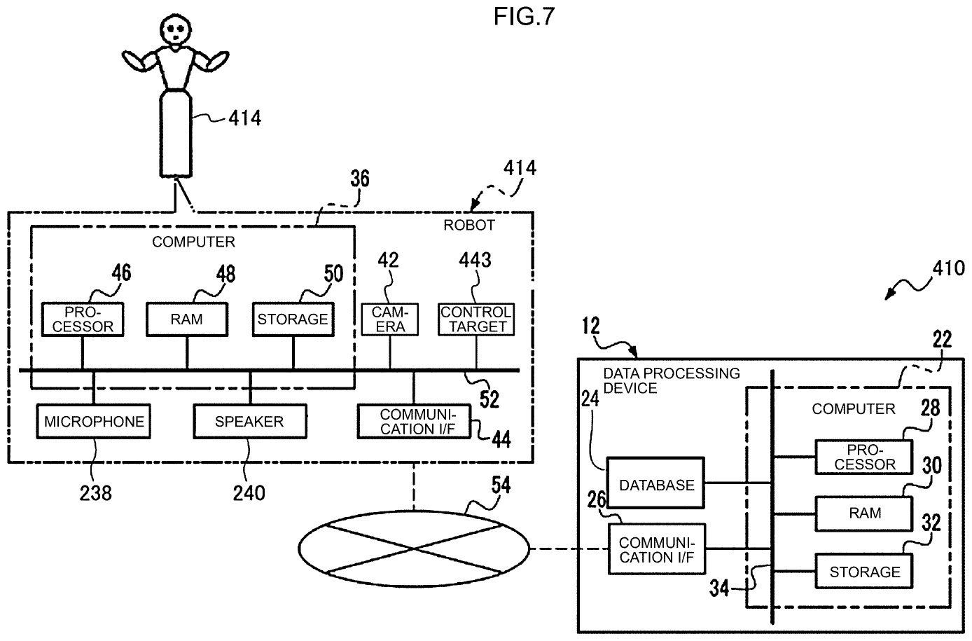

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

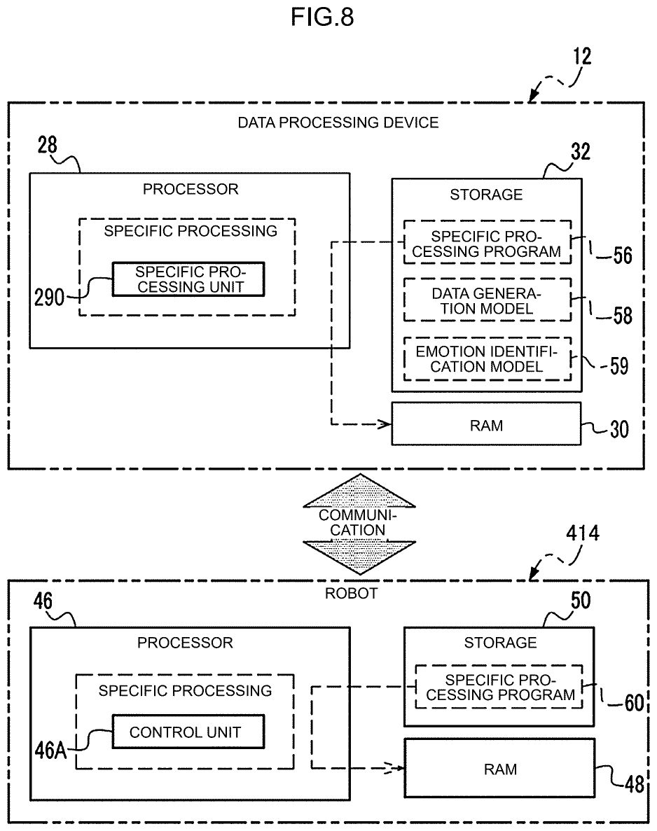

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the above-described collection unit, analysis unit, suggestion unit, and display unit is implemented by at least one of, for example, a robot 414 and a data processing apparatus 12. For example, the collection unit collects lifelog data and GPS data using a wearable device or smartphone of the robot 414. The analysis unit is implemented, for example, by a specific processing unit 290 of the data processing apparatus 12, and analyzes the data using AI to evaluate the user's physical condition. The suggestion unit is implemented, for example, by the specific processing unit 290 of the data processing apparatus 12, and proposes an optimal recipe to the user using AI. The display unit is implemented, for example, by a control unit 46A of the robot 414, and displays a price list of ingredients required for the proposed recipe. The correspondence between each unit and the device or control unit is not limited to the above examples and various modifications are possible.

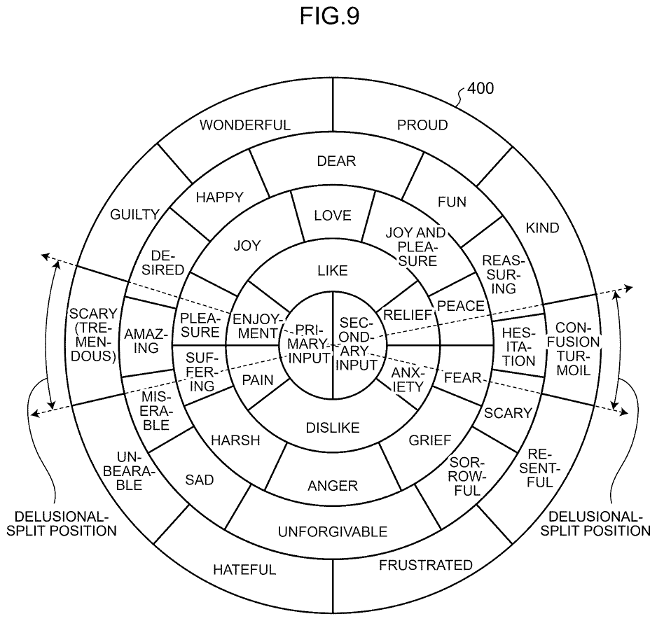

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

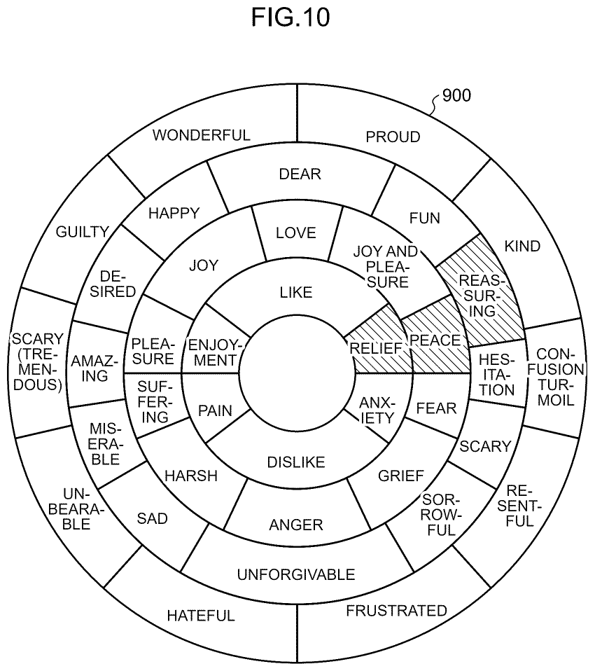

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1)A system comprising: a collection unit configured to collect lifelog data and GPS data; an analysis unit configured to analyze the data collected by the collection unit and evaluate the user's physical condition; a suggestion unit configured to propose a recipe based on the physical condition evaluated by the analysis unit; and a display unit configured to display a price list of ingredients required for the recipe proposed by the suggestion unit.
(Supplementary Note 2)The system according to Supplementary Note 1, wherein the collection unit is configured to collect lifelog data including sleep duration, amount of exercise, meal contents, heart rate, and blood pressure.
(Supplementary Note 3)The system according to Supplementary Note 1, wherein the display unit is configured to collect and display online price information.
(Supplementary Note 4)The system according to Supplementary Note 1, wherein the display unit is configured to collect and display price information in cooperation with a store database.
(Supplementary Note 5)The system according to Supplementary Note 1, wherein the suggestion unit is configured to collect menu information of restaurants within a radius of 500 meters from the current location when going out, and to propose restaurants that provide menus suitable for the user's physical condition.
(Supplementary Note 6)The system according to Supplementary Note 1, wherein the suggestion unit is configured to provide a mechanism for awarding points when a healthy meal is consumed.
(Supplementary Note 7)The system according to Supplementary Note 1, wherein the collection unit is configured to estimate the user's emotion and adjust the timing of lifelog data collection based on the estimated emotion of the user.
(Supplementary Note 8)The system according to Supplementary Note 1, wherein the collection unit is configured to analyze the user's past lifelog data and select a collection method.
(Supplementary Note 9)The system according to Supplementary Note 1, wherein the collection unit is configured to perform filtering based on the user's current living situation (for example, employment status, family environment) and areas of interest (for example, hobbies, interests) at the time of lifelog data collection.
(Supplementary Note 10)The system according to Supplementary Note 1, wherein the collection unit is configured to estimate the user's emotion and determine the priority of lifelog data to be collected based on the estimated emotion of the user.
(Supplementary Note 11)The system according to Supplementary Note 1, wherein the collection unit is configured to preferentially collect highly relevant data within a radius of 1 kilometer from the user's current location by considering the user's geographic location information at the time of lifelog data collection.
(Supplementary Note 12)The system according to Supplementary Note 1, wherein the collection unit is configured to analyze the user's social media activity and collect relevant data at the time of lifelog data collection.
(Supplementary Note 13)The system according to Supplementary Note 1, wherein the analysis unit is configured to estimate the user's emotion and adjust the criteria for physical condition evaluation based on the estimated emotion of the user.
(Supplementary Note 14)The system according to Supplementary Note 1, wherein the analysis unit is configured to improve the accuracy of physical condition evaluation by considering interrelationships among lifelog data (for example, the relationship between sleep duration and amount of exercise) during analysis.
(Supplementary Note 15)The system according to Supplementary Note 1, wherein the analysis unit is configured to refer to the user's past physical condition data during analysis to perform physical condition evaluation.
(Supplementary Note 16)The system according to Supplementary Note 1, wherein the analysis unit is configured to estimate the user's emotion and adjust the order of displaying the results of physical condition evaluation based on the estimated emotion of the user.
(Supplementary Note 17)The system according to Supplementary Note 1, wherein the analysis unit is configured to perform physical condition evaluation by considering the user's geographic distribution (for example, differences between urban and suburban areas) during analysis.
(Supplementary Note 18)The system according to Supplementary Note 1, wherein the analysis unit is configured to refer to relevant literature of the user during analysis to improve the accuracy of physical condition evaluation.
(Supplementary Note 19)The system according to Supplementary Note 1, wherein the suggestion unit is configured to estimate the user's emotion and adjust the expression method of recipe suggestions based on the estimated emotion of the user.
(Supplementary Note 20)The system according to Supplementary Note 1, wherein the suggestion unit is configured to adjust the level of detail of suggestions based on the user's physical condition when proposing recipes.
(Supplementary Note 21)The system according to Supplementary Note 1, wherein the suggestion unit is configured to apply different suggestion algorithms according to the user's meal history when proposing recipes.
(Supplementary Note 22)The system according to Supplementary Note 1, wherein the suggestion unit is configured to estimate the user's emotion and adjust the length of recipe suggestions based on the estimated emotion of the user.
(Supplementary Note 23)The system according to Supplementary Note 1, wherein the suggestion unit is configured to determine the priority of suggestions based on the user's meal history when proposing recipes.
(Supplementary Note 24)The system according to Supplementary Note 1, wherein the suggestion unit is configured to adjust the order of suggestions based on relevance to the user when proposing recipes.
(Supplementary Note 25)The system according to Supplementary Note 1, wherein the display unit is configured to estimate the user's emotion and adjust the display method of the price list based on the estimated emotion of the user.
(Supplementary Note 26)The system according to Supplementary Note 1, wherein the display unit is configured to select a display method by referring to the user's past purchase history when displaying the price list.
(Supplementary Note 27)The system according to Supplementary Note 1, wherein the display unit is configured to customize the display content based on the user's current living situation when displaying the price list.
(Supplementary Note 28)The system according to Supplementary Note 1, wherein the display unit is configured to estimate the user's emotion and determine the priority of the price list based on the estimated emotion of the user.
(Supplementary Note 29)The system according to Supplementary Note 1, wherein the display unit is configured to select a display method by considering the user's geographic location information when displaying the price list.
(Supplementary Note 30)The system according to Supplementary Note 1, wherein the display unit is configured to analyze the user's social media activity and propose display content when displaying the price list.

What is claimed is:

1. A system comprising:
circuitry configured to:
receive, from a client terminal via a packet-switched network, a plurality of time-series data vectors transmitted from a sensor of the client terminal, each time-series data vector representing a respective measurement category;
generate, by inputting the plurality of time-series data vectors as a multidimensional input tensor into a trained neural network comprising at least one of a convolutional neural network, a recurrent neural network, or a Transformer-based model, a plurality of numerical output scores;
generate, by inputting the plurality of numerical output scores into a data generation model obtained by deep learning on a neural network, inference data comprising a data record selected based on the plurality of numerical output scores; and
transmit the inference data to the client terminal via the packet-switched network.

2. The system according to claim 1, wherein the plurality of time-series data vectors comprise at least one of sleep duration data, exercise amount data, meal content data, heart rate data, or blood pressure data collected from a wearable device of the client terminal.

3. The system according to claim 1, wherein the circuitry is further configured to receive, from the client terminal, location data comprising latitude, longitude, and timestamp values, and retrieve, from a database based on the inference data and the location data, resource data comprising price information associated with the data record, and transmit the resource data to the client terminal via the packet-switched network.

4. The system according to claim 3, wherein the circuitry is further configured to retrieve the resource data from an external data source via an application programming interface in real time based on a geographic area determined from the location data.

5. The system according to claim 3, wherein the circuitry is further configured to collect menu information from a facility database based on the location data, analyze the menu information using a natural language processing model, and generate a recommendation list comprising facilities associated with data records matching the plurality of numerical output scores.

6. The system according to claim 1, wherein the circuitry is further configured to estimate an emotion of a user of the client terminal based on at least one of facial image data, audio data, or text data received from the client terminal by inputting the at least one of facial image data, audio data, or text data into an emotion identification model, and adjust a timing of receiving the plurality of time-series data vectors based on the estimated emotion.

7. The system according to claim 6, wherein the circuitry is further configured to, when the estimated emotion indicates a first emotional state, concentrate collection of the plurality of time-series data vectors during a first time period, and when the estimated emotion indicates a second emotional state, concentrate collection of the plurality of time-series data vectors during a second time period different from the first time period.

8. The system according to claim 1, wherein the circuitry is further configured to analyze past time-series data vectors stored in a database associated with a user of the client terminal using a recurrent neural network or a Transformer-based time-series pattern recognition model to extract behavioral patterns, and select a collection method for the plurality of time-series data vectors based on the extracted behavioral patterns.

9. The system according to claim 1, wherein the circuitry is further configured to perform filtering of the plurality of time-series data vectors based on attribute information of a user of the client terminal, the attribute information comprising at least one of an employment status, a family environment, or an area of interest.

10. The system according to claim 1, wherein the trained neural network receives, as the multidimensional input tensor, a three-dimensional tensor comprising a user dimension, a time dimension, and a feature dimension, and outputs the plurality of numerical output scores comprising at least one of a fatigue score, an estimated stress level, or a recommended nutrient category.

11. The system according to claim 1, wherein the circuitry is further configured to analyze interrelationships among the plurality of time-series data vectors by applying at least one of correlation analysis, co-occurrence network analysis, or a multivariate analysis model to detect a cross-category pattern across different measurement categories.

12. The system according to claim 1, wherein the circuitry is further configured to analyze historical time-series data vectors associated with a user of the client terminal stored in a database, detect a temporal trend in the historical time-series data vectors, and adjust the plurality of numerical output scores based on the detected temporal trend.

13. The system according to claim 1, wherein the circuitry is further configured to refer to an external literature database during generation of the plurality of numerical output scores, extract findings from the external literature database using a natural language processing model, and adjust an evaluation weight for at least one of the plurality of time-series data vectors based on the extracted findings.

14. The system according to claim 1, wherein the circuitry is further configured to estimate an emotion of a user of the client terminal by inputting at least one of facial image data, audio data, or text data into an emotion identification model, and adjust a method of expressing the inference data based on the estimated emotion, such that when the estimated emotion indicates stress, the inference data is generated in a simplified format, and when the estimated emotion indicates relaxation, the inference data is generated in a detailed format.

15. The system according to claim 1, wherein the circuitry is further configured to adjust a level of detail of the inference data based on the plurality of numerical output scores, such that when a first numerical output score exceeds a first threshold, the inference data is generated with a reduced number of procedural steps and a shortened description, and when a second numerical output score exceeds a second threshold, the inference data is generated with an increased number of procedural steps and supplementary information.

16. The system according to claim 1, wherein the circuitry is further configured to apply different selection algorithms to select the data record based on a history of past data records associated with a user of the client terminal, the different selection algorithms comprising at least one of collaborative filtering, content-based filtering, or reinforcement learning-based selection.

17. The system according to claim 1, wherein the circuitry is further configured to determine, based on the inference data, that a user of the client terminal has achieved a target condition indicated by the plurality of numerical output scores, and award points to an account associated with the user in response to determining achievement of the target condition.

18. A system comprising:
a communication interface configured to communicate with a client terminal via a packet-switched network, the client terminal comprising at least one of a wearable device, a smart device, smart glasses, a headset-type terminal, or a robot;
a processor comprising at least one of a central processing unit, a graphics processing unit, a general-purpose computing on graphics processing unit, an accelerated processing unit, or a tensor processing unit;
a random-access memory;
a memory storing a data generation model obtained by deep learning on a neural network and an emotion identification model;
a database; and
circuitry configured to:
receive, from the client terminal via the communication interface, a plurality of time-series data vectors collected from a sensor of the client terminal, each time-series data vector representing a respective measurement category, and location data comprising latitude, longitude, and timestamp values;
preprocess the plurality of time-series data vectors into a multidimensional input tensor stored in the random-access memory, the multidimensional input tensor comprising a user dimension, a time dimension, and a feature dimension;
generate, by inputting the multidimensional input tensor into a trained neural network comprising at least one of a convolutional neural network, a recurrent neural network, or a Transformer-based time-series analysis model, a plurality of numerical output scores comprising at least one of a fatigue score, a stress level, or a recommended nutrient category;
estimate an emotion of a user of the client terminal by inputting at least one of facial image data, audio data, or text data received from the client terminal into the emotion identification model stored in the memory;
generate, by inputting the plurality of numerical output scores into the data generation model stored in the memory, inference data comprising a data record selected from the database based on the plurality of numerical output scores;
retrieve, from the database based on the inference data and the location data, resource data comprising price information obtained from a store information source within a geographic area determined from the location data; and
transmit, via the communication interface, the inference data and the resource data to the client terminal via the packet-switched network, wherein a display format of the inference data is adjusted based on the estimated emotion.

19. The system according to claim 18, wherein the circuitry is further configured to collect menu information from a facility database based on the location data, analyze the menu information using a natural language processing model comprising a large language model, and generate a recommendation list comprising facilities associated with data records matching the plurality of numerical output scores, and transmit the recommendation list to the client terminal via the communication interface.

20. A method performed by circuitry of a system, the method comprising:
receiving, from a client terminal via a packet-switched network, a plurality of time-series data vectors transmitted from a sensor of the client terminal, each time-series data vector representing a respective measurement category;
generating, by inputting the plurality of time-series data vectors as a multidimensional input tensor into a trained neural network comprising at least one of a convolutional neural network, a recurrent neural network, or a Transformer-based model, a plurality of numerical output scores;
generating, by inputting the plurality of numerical output scores into a data generation model obtained by deep learning on a neural network, inference data comprising a data record selected based on the plurality of numerical output scores; and
transmitting the inference data to the client terminal via the packet-switched network.
